(19) United States
(12) Patent Application Publication
(10) Pub. No.: US 2024/0423880 A1
(43) Pub. Date: Dec. 26, 2024

(54) FLUSHABLE WET WIPES

(71) Applicant: The University of Toledo, Toledo, OH (US)

(72) Inventors: Yakov Lapitsky, Toledo, OH (US); Kunal Choudhuri, Toledo, OH (US); Umberto A. Kober, Toledo, OH (US)

(73) Assignee: The University of Toledo, Toledo, OH (US)

(21) Appl. No.: 18/746,938

(22) Filed: Jun. 18, 2024

Related U.S. Application Data
(60) Provisional application No. 63/522,005, filed on Jun. 20, 2023.

(51) Int. Cl. A61K 8/02; A61K 8/73; A61Q 19/10

(52) U.S. Cl. CPC A61K 8/0208; A61K 8/73; A61Q 19/10; A61K 2800/30; A61K 2800/56

(57) ABSTRACT

Flushable wet wipes, and methods of making and using the same, are described. The flushable wet wipes include a biopolymer comprising a carrageenan and optionally a monovalent or multivalent salt on a substrate.

### RELATED APPLICATIONS

This application claims priority to U.S. Provisional Application No. 63/522,005 filed under 35 U.S.C. § 111(b) on Jun. 20, 2023, the disclosure of which is incorporated herein by reference in its entirety.

### STATEMENT REGARDING FEDERALLY SPONSORED RESEARCH

This invention was made with government support under grant number 84014801 awarded by the Environmental Protection Agency. The government has certain rights in this invention.

### BACKGROUND

Wet wipes, also known as moist wipes or wet napkins (such as baby wipes), are a type of widely used disposable personal hygiene product that are used for cleaning and refreshing various parts of the body. Wet wipes are a carefully designed personal hygiene product that are intended to provide effective cleaning and refreshing while also being gentle on the skin. The composition and structure of wet wipes are engineered to provide softness, absorbency, and strength while also incorporating additional ingredients to provide other benefits. The convenience and ease of use of wet wipes make them a popular choice for personal hygiene and cleaning tasks. However, it is important to dispose of wet wipes properly to avoid environmental damage.

Wet wipes are widely used for personal cleaning and homecare and are often flushed in a toilet. However, flushing wet wipes, even those marketed as “flushable”, has (in the case of many tested products) been shown to clog pipes and pumps in wastewater collection and treatment systems, especially after agglomeration with other organic/inorganic matter. Even “flushable” wipes have frequently been found to exhibit poor dispersibility, leading to high maintenance costs for municipalities. Flushable wipes have been prepared previously from viscose/regenerated cellulose fibers, or from binding the wipe together with a polymeric binder that remains insoluble at high salt concentration but dissolves when their salt concentration decreases upon flushing. However, despite these approaches, products in the market (with relatively few exceptions) fail flushability tests.

Wipe binders that dissociate upon flushing have also been achieved by precipitating hydrophobic synthetic polyelectrolytes with salts. However, such binders are not biopolymer-based, and such wipes are not paper-based, making these binders less than ideal from an environmental perspective.

Also, many commercial wet wipes contain synthetic plastic fibers (i.e., microplastics) that contribute to water pollution. The presence of plastic fibers as binder components in conventional wet wipes contributes to water pollution through microplastics release. With environmental regulations progressively becoming more stringent, there is increasing industrial interest in developing alternative, plastic-free wet wipes.

To overcome these limitations, there is a need in the art for synthetic plastic-free wipes that are truly flushable (i.e., that rapidly disperse upon being flushed).

### SUMMARY

Provided is a wet wipe comprising a water-soluble or dispersible substrate; and a biopolymer hydrogel composition interspersed on, infused into, or incorporated into, the water-soluble or dispersible substrate, wherein the biopolymer hydrogel composition comprises kappa-carrageenan; wherein the wet wipe is configured to disperse upon immersion in water with agitation.

In certain embodiments, the biopolymer hydrogel composition is interspersed on, infused into, or incorporated into, a plurality of layers of the water soluble or dispersible substrate.

In certain embodiments, the biopolymer hydrogel composition further comprises a solvent. In particular embodiments, the solvent comprises water, ethanol, acetic acid, dimethyl sulfoxide (DMSO), or polyethylene glycol (PEG). In particular embodiments, the kappa-carrageenan is present in the biopolymer hydrogel composition in an amount of less than 50% by weight.

In certain embodiments, the biopolymer hydrogel composition further comprises one or more of iota-carrageenan, alginate, chitosan, cellulose, starch, gelatin, xanthan gum, hyaluronic acid, pectin, poly-lactic acid (PLA), chondroitin sulfate, collagen, elastin, peptidoglycan, or derivatives thereof.

In certain embodiments, the biopolymer hydrogel composition comprises one or more crosslinkers.

In certain embodiments, the water-soluble or dispersible substrate is a paper substrate. In certain embodiments, the water soluble or dispersible substrate is a non-woven fabric. In particular embodiments, the non-woven fabric comprises toilet paper, tissue paper, cotton, or rayon.

In certain embodiments, the wet wipe comprises two layers of the water-soluble or dispersible substrate. In certain embodiments, the water-soluble or dispersible substrate consists of a single layer. In certain embodiments, the wet wipe comprises three or more layers of the water-soluble or dispersible substrate.

In certain embodiments, the wet wipe further comprises a monovalent or multivalent salt in the biopolymer hydrogel composition. In particular embodiments, the monovalent or multivalent salt is present in the biopolymer hydrogel composition at a concentration ranging from about 1 mM to about 1000 mM. In particular embodiments, the monovalent or multivalent salt is present in the biopolymer hydrogel composition at a concentration ranging from about 10 mM to about 1000 mM. In particular embodiments, the monovalent or multivalent salt comprises KCl, NaCl, LiCl, NH₄Cl, NaBr, KBr, NaI, CaCl₂), ZnCl₂, CaCO₃, MgSO₄, sodium acetate, potassium acetate, ammonium acetate, sodium citrate, potassium citrate, ammonium citrate, calcium acetate, or calcium lactate.

In certain embodiments, the wet wipe further comprises one or more additives for cleansing, disinfecting, skin benefits, surface cleaning, or surface disinfection.

In certain embodiments, the biopolymer composition comprises kappa-carrageenan, a monovalent salt, and water. In particular embodiments, the water-soluble or dispersible substrate comprises toilet paper.

In certain embodiments, the wet wipe further comprises a surfactant, a pH adjustment agent, a preservative, or a fragrance.

In certain embodiments, the wet wipe is free of alcohol. In certain embodiments, the wet wipe is free of plastic. In certain embodiments, the wet wipe is free of alcohol and free of plastic.

In certain embodiments, the wet wipe has a tensile strength of about 10² N/m or more. In certain embodiments, the wet wipe has a tensile strength of at least about 50 N/m.

In particular embodiments, the water-soluble or water dispersible substrate comprises toilet paper.

In certain embodiments, the wet wipe is free of alcohol and free of plastic, and the water soluble or dispersible substrate comprises toilet paper.

Further provided is a flushable wet wipe comprising a first layer of toilet paper; a second layer of toilet paper; and a biopolymer hydrogel comprising a carrageenan between the first layer and the second layer or interspersed within the first layer and/or the second layer. In certain embodiments, the biopolymer hydrogel comprises kappa-carrageenan. In certain embodiments, the toilet paper is a multi-ply toilet paper. In certain embodiments, the toilet paper is a two-ply toilet paper. In certain embodiments, the biopolymer hydrogel comprises kappa-carrageenan.

Further provided is a biopolymer composition comprising less than 50% by weight kappa-carrageenan; from about 10 mM to about 1000 mM of a monovalent salt; and water.

Further provided is a method of making a flushable wet wipe, the method comprising adding a warm solution of a carrageenan to a plurality of sheets of toilet paper; allowing the carrageenan to infuse into the sheets of toilet paper for a period of time, or pressing the solution into the toilet paper, to create infused toilet paper; allowing the infused toilet paper to cool; and immersing the infused toilet paper in a solution of a monovalent salt to make a flushable wet wipe. In certain embodiments, the carrageenan is kappa-carrageenan. In certain embodiments, the period of time is about one minute. In certain embodiments, the period of time is less than one minute. In certain embodiments, the monovalent salt comprises KCl. In certain embodiments, the solution is pressed into the toilet paper until the solution is uniformly distributed and the toilet paper is flat and smooth.

Further provided is a method of making a flushable wet wipe, the method comprising overlaying a first layer of a water dispersible substrate crosswise on a second layer of the water dispersible substrate to form a layer stack; infusing the layer stack with a solution comprising kappa-carrageenan; spreading the solution on the layer stack by pressing the layer stack together; and immersing the layer stack in a salt solution to create a biopolymer hydrogel composition interspersed on, infused into, or incorporated into the layer stack and thereby make a flushable wet wipe. In certain embodiments, the water dispersible substrate is toilet paper. In certain embodiments, the salt solution comprises KCl. In certain embodiments, the layer stack is pressed together until the solution is uniformly distributed and the layer stack is flat and smooth.

Further provided is a kit for making a flushable wet wipe, the kit comprising a first container housing a water soluble or dispersible substrate material; and a second container housing a biopolymer composition comprising a carrageenan, a monovalent salt, and water.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Illustration depicting a flushable wet wipe turning into wet toilet paper upon immersion in water. The flushable wet wipe undergoes polysaccharide dissolution in excess water to leave behind a wet toilet paper sheet.

FIGS. 2A-2B: Illustrations of non-limiting example methods for making a flushable wet wipe using carrageenan (FIG. 2A) and an alginate-based alcohol wipe (FIG. 2B). FIGS. 2A-2B also include digital photographs of an alginate-based alcohol wipe prepared using a 3 wt % parent alginate solution and 3 layers of toilet paper (FIG. 2B) or a kappa-carrageenan-based wipe prepared using 3 wt % kappa-carrageenan solution, 2 layers of toilet paper, and a 100 mM KCl solution (FIG. 2A).

FIGS. 3A-3D: FIG. 3A shows a photograph showing wet toilet paper strips (of three sheets stacked together) easily ripping while the wet alcohol wipe created by ethanol-triggered gelation of 3 wt % alginate solution remains intact. FIG. 3B shows tensile strengths of alcohol wipes prepared using 1-3 wt % alginate solutions before (blue) and 10 min after (green) immersion in 4 L of water (mean±SD). The shaded region indicates the measured tensile strength of three stacked sheets of alginate-free, wet toilet paper (a band within an SD of the mean measurement). The shaded region in the background shows the strength of wet toilet paper. FIG. 3C shows images of an alcohol wipe (prepared with 3 wt % alginate solution) dispersing upon its gentle agitation in 4 L of water. While a commercial wet wipe (DUDE® Wipes) fails to disperse with this gentle agitation, the alginate and toilet paper-based alcohol wipe becomes weak and fragments into smaller pieces. FIG. 3D shows the FTIR spectra of (i) control toilet paper, (ii) sodium alginate, and (iii) alginate-based wet wipe 30 min after immersion in 4 L of water. For clarity, spectra (ii) and (iii) were offset upward by 0.2 and 0.4 absorbance units, respectively.

FIG. 4: Tensile strengths of alcohol wipes prepared using 1-3 toilet paper layers infused with 3 wt % alginate solution before and 10 min after immersion in 4 L of water (mean±SD). The dashed line indicates the target wet wipe strength of 100 N/m.

FIGS. 5A-5C: FIG. 5A shows tensile strengths of aqueous wet wipes prepared using two toilet paper layers (in strips) infused with 2-4 wt % kappa-carrageenan solutions before (blue) and 10 min after (green) immersion in 4 L of water. The shaded region in the background shows the strength of wet toilet paper. FIG. 5B shows the effects of the KCl concentration in the gelation medium on the tensile strength of the wipes prepared using 3 wt % kappa-carrageenan and two toilet paper layers (in strips) in solutions of various KCl concentrations (mean±SD). FIG. 5C shows the strength of alginate- and carrageenan-containing wet wipes over time from simulated flushing by showing the rates of tensile strength reduction upon the immersion in 4 L of water of wipes prepared using 3 wt % alginate (squares) and 3 wt % kappa-carrageenan (circles) parent solution concentrations (mean±SD) with two toilet paper layers (in strips). The lines are guides to the eye, while the shaded regions indicate the measured tensile strength of wet toilet paper (stacked into three overlapping layers).

FIGS. 6A-6B: Average sludge volume index (SVI) changes in bioreactors containing degradation products of alginate-strengthened alcohol wipes and kappa-carrageenan-strengthened aqueous wipes dispersed in 1 gal of water and diluted to either 5% (FIG. 6A) or 50% (FIG. 6B) of the initial 1-gal dispersion (mean±SD).

FIGS. 7A-7B: Chemical oxygen demand (COD) removal in bioreactors containing degradation products of alginate-strengthened alcohol wipes and kappa-carrageenan-strengthened aqueous wipes dispersed in 1 gal of water and diluted to either 5% (FIG. 7A) or 50% (FIG. 7B) of the initial 1-gal dispersion (mean±SD).

FIGS. 8A-8B: Mixed liquor volatile suspended solids (MLVSS) changes in bioreactors containing degradation products of alginate-strengthened alcohol wipes and kappa-carrageenan-strengthened aqueous wipes dispersed in 1 gal of water and diluted to either 5% (FIG. 8A) or 50% (FIG. 8B) of the initial 1-gal dispersion (mean±SD).

FIGS. 9A-9B: Biomass stress indices (BSI) changes in bioreactors containing degradation products of alginate-strengthened alcohol wipes and kappa-carrageenan-strengthened aqueous wipes dispersed in 1 gal of water and diluted to either 5% (FIG. 9A) or 50% (FIG. 9B) of the initial 1-gal dispersion (mean±SD).

FIG. 10: FTIR spectra of (i) control toilet paper, (ii) kappa-carrageenan, and (iii) kappa-carrageenan-based wet wipes 10 minutes after simulated flushing as a function of wavenumber. Spectra (ii) and (iii) were offset upward by 0.2 and 0.4 absorbance units, respectively, while the kappa-carrageenan concentration in the wipe corresponding to spectrum (iii) was 3 wt %.

FIG. 11: Confocal microscopy images before (left) and after (right) immersing an alginate-containing wet wipe in water, confirming the presence of the alginate in the fibers of the wipe. The presence of green fluorescently labeled alginate in the space surrounding the orange paper fibers both before (left image) and 10 minutes after (right image) immersion in 4 L of water was confirmed by the confocal laser-scanning microscopy (CLSM). The wipes used for this analysis were prepared using three toilet paper layers and 3 wt % alginate solution.

FIG. 12: Graphs showing that the change in tensile strength from simulated flushing (i.e., immersion in 4 L of water) occurs over a range of temperatures. The graphs show the rates of the wipe tensile strength reduction upon the water immersion of wipes prepared using (a) 3 wt % alginate and (b) 3 wt % kappa-carrageenan parent solutions (when immersed in 100 mM KCl) at 7° C. (▪), room temperature (●), and 40° C. ( [symbol] ) (mean±SD). The shaded regions indicate the measured tensile strengths of stacked three (left) and two (right) overlapping layers of wet toilet paper (and represent the range within one SD of the mean measurement). The tensile strength data for the 40° C. samples could not be taken at the 10 min time point as the wipes had already dispersed into pieces.

FIGS. 13A-13B: Graphs showing tensile strength of perforated (FIG. 13A) and non-perforated (FIG. 13B) carrageenan-containing wet wipes having different concentrations of carrageenan.

FIG. 14: Apparent G′ values obtained at to ω=1 rad/s from alginate alcogels prepared from variably concentrated parent alginate solutions (mean±SD).

FIGS. 15A-15D: Representative temperature sweeps performed on kappa-carrageenan gels prepared at 2 (□), 3 (∘), and 4 (⋄) wt % polymer concentrations containing 100 mM KCl (FIG. 15A), G′ values of 2, 3, and 4 wt % carrageenan gels prepared with 100 mM KCl at 25° C. (FIG. 15B), representative temperature sweeps performed on kappa-carrageenan gels prepared at the 3 wt % polymer concentration and with either 0 (□), 100 (∘), or 200 (⋄) mM KCl (FIG. 15C), and G′ values of gels prepared at 3 wt % kappa-carrageenan gels prepared with 0, 100, and 200 mM KCl 25° C. (FIG. 15D). The error bars in FIGS. 15B, 15D are standard deviations.

FIG. 16: Variation in the wet wipe tensile strength with number of base toilet paper layers for water/kappa-carrageenan-based wipes prepared from 2 and 3 wt % kappa-carrageenan (mean±SD).

FIGS. 17A-17D: Digital photographs of alginate-based alcohol wipes prepared using a 3 wt % parent alginate solution and 3 layers of toilet paper (FIGS. 17A, 17C) and kappa-carrageenan-based wipes prepared using 3 wt % parent kappa-carrageenan solution, 2 layers of toilet paper, and 100 mM KCl solution (FIGS. 17B, 17D). The smaller samples (FIGS. 17A, 17B) were prepared using toilet paper strips, while the larger, full-size samples (FIGS. 17C, 17D) were prepared using nearly full toilet paper squares.

FIG. 18: Schematic illustrating the preparation of embossed flushable wipes: at step (i), K-carrageenan solution was first applied to the toilet paper stacks in a stripe-like pattern; at step (ii), the K-carrageenan solution-covered toilet paper was then inserted into 5″×7″ plastic embossing folders; and at step (iii), the folders were then passed through a rolling press, resulting in K-carrageenan solution-infused toilet paper with an embossed pattern, shown at step (iv). The final wet wipes were then generated by immersing these embossed toilet paper stacks in KCl solution.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein are wet wipes that rapidly disintegrate/disperse when flushed, and in some embodiments are plastic-free. In accordance with the present disclosure, by incorporating a biopolymer hydrogel into layers of a flushable paper substrate such as toilet paper, wet wipes which have good tensile strength but disintegrate in the presence of excess water can be produced. By infusing regular toilet paper with aqueous biopolymer solutions followed by gelation, wet wipes can be created that possess tensile strengths comparable to commercial flushable wet wipes. Upon simulated flushing, the hydrogel network breaks down, and within minutes, the wet wipe tensile strength drops to values obtained for wet toilet paper. Moreover, the tensile strength can be tuned by adjusting the biopolymer concentrations in the formulations. This rapid drop in tensile strength upon flushing can prevent accumulation of wet wipes in the wastewater pipes, thus preventing clogging, while the use of biopolymers in the wet wipes reduces the accumulation of harmful plastics in the environment.

In general, the wet wipes include a water-soluble or dispersible substrate and a biopolymer composition, where the biopolymer composition is interspersed, infused, and/or coated on the substrate. The substrate may be a paper substrate. The substrate may be a non-woven fabric such as toilet paper or tissue paper. The substrate may be made from wood pulp or recycled paper fibers through known papermaking methods, and may be absorbent to ensure effective cleaning and flushability. The substrate may be made, for example, by forming a paper pulp into thin sheets using a papermaking machine, embossing or perforating the thin sheets to provide texture and absorbency, and cutting the sheets into a desired size. However, other water soluble or dispersible substrates are possible and encompassed within the scope of the present disclosure. For example, the substrate may be composed of a non-woven material made from natural fibers, such as cotton or rayon (which is a semi-synthetic fiber made from natural cellulose materials, such as wood pulp, cotton, and bamboo).

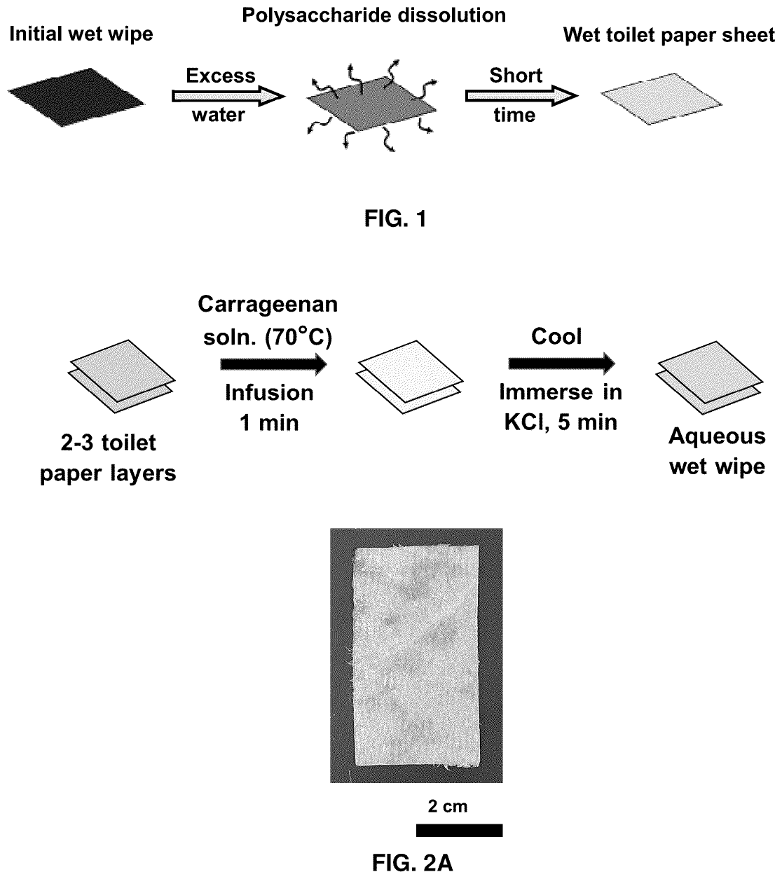

The wet wipes can be based on toilet paper, which is easily dispersible upon flushing, and abundant and biodegradable biopolymer-based gels. Toilet paper can be reinforced with a natural biopolymer (polysaccharide) gel that can be triggered to dissolved when the wipe is immersed in excess water (FIG. 1). The gel dissolution leaves behind truly flushable/dispersible toilet paper sheets and produces no microplastics. Rather, the wet wipe includes a natural, non-toxic biopolymer and some salt. Water-based flushable wet wipes can be prepared by strengthening/binding regular wet wipes with certain types of hydrogels. Since these hydrogels can contain no alcohol and transform into non-toxic biopolymer solutions upon flushing, this technology can be both alcohol- and plastic-free.

In some embodiments, the substrate is a plurality of layers of toilet paper. The wet wipes can incorporate biopolymers to strengthen regular toilet paper so as to possess tensile strength comparable to commercial flushable wet wipes and still be dispersible upon immersion in water, especially upon immersion in water with agitation (such as that which occurs from flushing). Toilet paper is a type of non-woven fabric. Non-woven materials are fabrics which are created by bonding fibers together through various techniques, such as heat, pressure, or chemical bonding, rather than weaving and knitting them together. Toilet paper is made by pressing and drying a mixture of wood fibers and other materials, such as recycled paper, to create a non-woven material that is soft, absorbent, and disposable. Toilet paper is a widely-used consumer product that is soft, absorbent, and disposable tissue paper primarily designed for hygiene purposes. The composition and structure of toilet paper have evolved over the years, but the fundamental characteristics remain the same. In its basic form, toilet paper is made from a blend of softwood and hardwood fibers, which are pulped, bleached, and pressed into thin sheets. The fibers used in toilet paper production are typically sourced from managed forests, recycled paper, or a combination of both. These fibers are refined to create a fluffy and soft texture, which provides comfort and absorption when used. The structure of toilet paper is designed to balance the need for strength and absorbency while maintaining a soft and comfortable texture. Toilet paper is typically made up of two or more layers of thin sheets, which are bonded together using a variety of techniques such as embossing, lamination, or gluing. These layers are engineered to provide strength and durability while maintaining a soft and fluffy texture that does not irritate sensitive skin. The surface of toilet paper may be treated with a variety of materials, such as lotions or oils, to provide a soothing and moisturizing effect. The texture of the paper can also be adjusted by varying the thickness and weight of the individual sheets or by embossing the surface to create a pattern or design.

A single layer of the water-soluble or dispersible substrate, or a plurality of layers of the water-soluble or dispersible substrate, may be included in the wet wipes. In some examples, the wet wipes include two layers of the water-soluble or dispersible substrate. In other examples, the wet wipes include three layers of the paper substrate. While the number of layers of the water-soluble or dispersible substrate is not particularly limited, increasing the number of layers of the water-soluble or dispersible substrate may reduce the flushability of the wet wipe. Therefore, the number of layers of water-soluble or dispersible substrate in the wet wipes may typically range from 1 to 10. However, wet wipes having more than 10 layers of water-soluble or dispersible substrate are nonetheless encompassed within the scope of the present disclosure.

The biopolymer composition includes a biopolymer and may be a hydrogel, which is a network formed by crosslinking or self-assembly of biopolymers that can absorb and retain large amounts of water or biological fluids. The biopolymer can be used to mechanically strengthen the substrate, and bind two or more substrate layers together, all while allowing for disintegration of the wet wipe upon immersion in water. Suitable biopolymers include, but are not limited to, carrageenans (kappa or iota), alginate, chitosan, cellulose, starch, gelatin, xanthan gum, hyaluronic acid, pectin, poly-lactic acid (PLA), chondroitin sulfate, collagin, elastin, peptidoglycan, or derivatives thereof. It has been found that kappa-carrageenan is particularly beneficial because it allows for the creation of alcohol-free flushable wet wipes.

The biopolymer composition may include combinations of two or more of the biopolymers. Thus, for example, the biopolymer composition may include kappa-carrageenan and one or more additional biopolymers such as, but not limited to, iota-carrageenan, alginate, chitosan, cellulose, starch, gelatin, xanthan gum, hyaluronic acid, pectin, poly-lactic acid (PLA), chondroitin sulfate, collagin, elastin, peptidoglycan, or derivatives thereof. The biopolymer(s) may be present in the biopolymer composition in an amount ranging from about 0.5 wt % to about 10 wt %. The biopolymer composition may further include one or more solvents such as, but not limited to, water, ethanol, acetic acid, dimethyl sulfoxide (DMSO), glycerol, or polyethylene glycol (PEG). Optionally, the biopolymer composition may further include one or more crosslinkers, such as calcium ions, glutaraldehyde, carbodiimides, genipin, or enzymes.

The biopolymer composition may also include one or more modifiers for increasing the stiffness or strength of the biopolymer composition. Modifiers include, but are not limited to, monovalent salts such as, but not limited to, KCl, NaCl, LiCl, NH₄Cl, NaBr, KBr, or NaI, or multivalent salts such as, but not limited to, CaCl₂), ZnCl₂, CaCO₃, MgSO₄, sodium acetate, potassium acetate, ammonium acetate, sodium citrate, potassium citrate, ammonium citrate, calcium acetate, or calcium lactate. For example, at low concentrations, KCl can increase the stiffness and strength of a carrageenan hydrogel because KCl ions can interact with the carrageenan molecules to enhance the intermolecular association between the carrageenan chains, which leads to a stronger and more stable gel structure.

In some embodiments, the biopolymer composition includes carrageenan. Carrageenan is a naturally occurring polysaccharide extracted from red seaweed. Carrageenan is a water-soluble, gel-forming substance that is commercially used to stabilize and thicken food products. Carrageenan is generally recognized as safe (GRAS) by the United States Food and Drug Administration (FDA).

There are two main types of carrageenan: kappa and iota. Kappa-carrageenan forms a strong, rigid gel while iota-carrageenan forms a softer, more elastic gel. The difference in gel strength is due to the difference in the chemical structure of the carrageenan molecules. Kappa-carrageenan has a higher degree of sulfation than iota-carrageenan, which allows it to form a more rigid gel structure. As shown in the examples herein, wet wipes made with kappa-carrageenan exhibit advantageous strength and flushability properties.

Carrageenan has previously been used in wipes, in much lower concentrations, as a cleaner rather than a binder. However, carrageenan by itself is insufficient for producing a strengthening effect. Carrageenan has also previously been used as a binder in dry paper or as the main components of wipes with less than 10% moisture content. However, neither technology utilized the carrageenan binders in a wet/hydrogel state, or a wet wipe. Carrageenan has also been used in detergent formulations (that can be used on surfaces together with wipes) where the formulations use surface-cleaning gel particles rather than continuous gels as binders for paper wipes. Carrageenan has also been used in the gel state to control liquid migration in wipes, which do not use carrageenan as a binder, or with paper.

In some embodiments, the biopolymer composition includes alginate. Alginate is a water-soluble polysaccharide commonly used as a food additive. As shown in the examples herein, wet wipes made with alginate exhibit useful strength and flushability properties. However, alginate wipes include an alcohol such as ethanol, which may not be ideal for certain applications. In other embodiments, such as embodiments containing kappa-carrageenan instead of alginate, the wet wipe can be free of alcohol.

The wet wipes may further include one or more additives for cleansing, disinfecting, or skin benefits, or pH adjustment agents, preservatives, or fragrances. The additives may also include additives for surface cleansing, homecare, or healthcare applications. The wet wipes may be particularly useful in a hospital or other healthcare setting, for example to cleanse patients unable to bathe themselves. The additives can be infused into the wet wipes or included as part of the biopolymer composition. For example, the additives may enhance performance or provide additional benefits to the user. These additives can include natural or synthetic ingredients that are designed to provide moisturizing, soothing, cleansing, or deodorizing effects. Possible additives include, but are not limited to, aloe vera, vitamin E, chamomile, tea tree oil, witch hazel, coconut oil, shea butter, fragrances, glycerin, soaps, cleaning agents, preservatives, chelating agents, pH adjusters, and disinfectants.

Aloe vera is a natural plant extract that is known for its moisturizing and soothing properties. Aloe vera is often added to wet wipes to help soothe and hydrate the skin. Vitamin E is an antioxidant that is commonly added to wet wipes to help protect the skin from damage caused by free radicals. Vitamin E can also help to moisturize and soothe the skin. Chamomile is a natural plant extract that is known for its calming and soothing effects on the skin. Chamomile can be added to the wet wipes to help reduce redness and irritation. Tea tree oil is a natural essential oil that is known for its antibacterial and antifungal properties. Tea tree oil can be added to the wet wipes to help kill germs and prevent infection. Witch hazel is a natural astringent that is known for its ability to reduce inflammation and redness on the skin. Witch hazel can be added to the wet wipes to help soothe, cleanse, and tone the skin. Fragrances can be added to the wet wipes to provide a pleasant scent and enhance the user's experience. However, it is important to note that some people may be sensitive to fragrances and may experience skin irritation. Glycerin is a natural humectant that is known for its ability to attract and retain moisture in the skin. Glycerin can be added to the wet wipes to help hydrate and soften the skin. Coconut oil is a natural moisturizer that can help provide hydration to the skin. Shea butter is a rich moisturizer that can help to protect and nourish the skin.

There are many types of disinfectants which can be incorporated into the wet wipes, including certain surfactants. Non-limiting examples include isopropyl alcohol, hydrogen peroxide, quaternary ammonium compounds, chlorhexidine, and benzalkonium chloride.

Furthermore, the wet wipes may also include one or more surfactants (whether disinfectants or not), moisturizers, or preservatives that are either infused into the wet wipes or included as part of the biopolymer composition. Surfactants may help emulsify, disperse, and remove dirt, oil, and other contaminants from the surface being cleaned with the wet wipes. Non-limiting example surfactants include sodium lauryl sulfate (SLS), cocamidopropyl betaine, polysorbate 20, cetylpyridinium chloride, and sodium cocoamphoacetate. However, many other surfactants are possible and encompassed within the scope of the present disclosure. When the wet wipes include a surfactant, the wet wipes may effectively remove dirt, bacteria, and other contaminants from skin without causing irritation or discomfort.

There are many types of soaps which can be incorporated into the wet wipes to enhance cleaning and sanitizing. Non-limiting example soaps include antibacterial soaps, baby soap, tea trea oil soap, castile soap, and sulfate-free soap.

Moisturizers may help prevent a user's skin from drying out after using the wet wipes. Non-limiting example moisturizers which may be included in the wet wipe compositions include glycerin, aloe vera, panthenol, sodium PCA, hyaluronic acid, and shea butter.

Preservatives may be included in the wet wipe composition to help prevent microbial growth and keep the wet wipes safe for use. Non-limiting example preservatives which may be included in the wet wipe composition include benzalkonium chloride, phenoxyethanol, methylisothiazolinone, chlorhexidine digluconate, potassium sorbate, and sodium benzoate.

pH adjusters may be used to maintain an optimal or useful pH level for the wet wipes. pH adjusters can be added to ensure compatibility with different surfaces. Suitable pH adjusters include, but are not limited to, citric acid or sodium hydroxide.

Chelating agents may be included to improve the effectiveness of other ingredients in the wet wipes by, for example, binding to metal ions that may be present in water or on surfaces. One non-limiting example of a suitable chelating agent is ethylene diamine tetraacetic acid (EDTA). However, many other chelating agents are possible and encompassed within the scope of the present disclosure.

Tactile properties are physical characteristics such as texture, roughness, smoothness, hardness, elasticity, and stickiness. The tactile properties of the wet wipes can be modified through any of a variety of ways. For example, the type of fiber used to make the wet wipe, its density, and its orientation can all affect the tactile properties of the wet wipe. Using a more porous fiberous substrate may increase the absorbency and softness of the wet wipe. The surface texture and chemistry of the wet wipe can also be changed by adding a surface treatment or coating to the wet wipe to make it feel smoother or more textured. As another example, the liquid used to saturate the wet wipe can be changed or modified, such as by adding a surfactant to affect the slipperiness and lubricity of the wet wipe. As another example, changing the pressure or temperature during manufacturing can affect the softness or stiffness of the wet wipe.

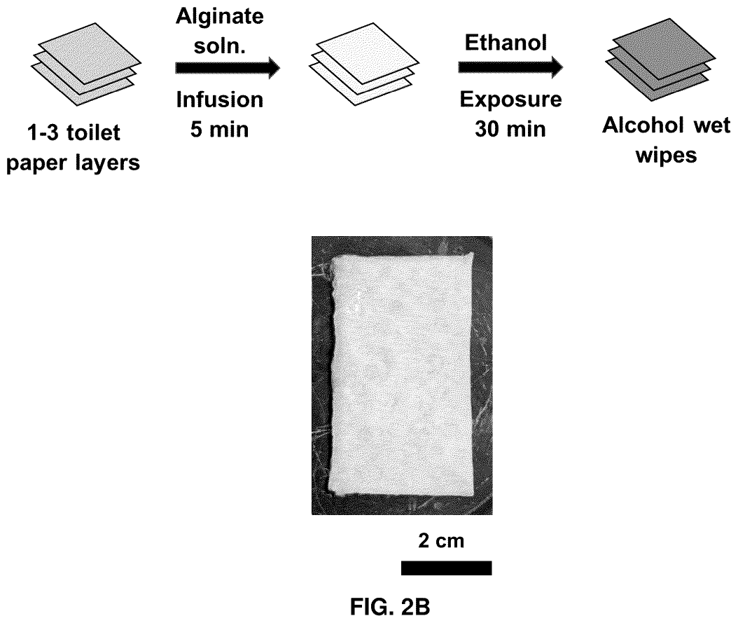

The wet wipes can be prepared through any method that satisfactorily incorporates the biopolymer composition into the paper substrate so as to render the resulting wet wipe flushable. Two alternative methods for making the wet wipes are depicted in FIGS. 2A-2B.

FIG. 2A depicts a non-limiting example method for making a flushable wet wipe. In the non-limiting example method, two or three layers of toilet paper are infused with a carrageenan solution at an elevated temperature for a period of time, such as about 70° C. for about 1 minute, to produce an infused paper product which is then allowed to cool and immersed in a salt solution for a period of time, such as a KCl solution for about 5 minutes, to produce an aqueous wet wipe that is flushable. This method for making a flushable wet wipe is conducive to a continuous production process. For example, the process may include a continuous curtain-coating by kappa-carrageenan solution followed by KCl solution immersion and calendaring. However, it is understood that faster biopolymer and KCl infusion can be achieved with process optimization, and such process optimization is within the scope of the present disclosure.

FIG. 2B depicts an alternative method for making a flushable wet wipe. This method involves infusing toilet paper with an alginate solution followed by precipitation in ethanol, resulting in alcohol-containing wet wipes. While these wet wipes may be flushable and plastic-free, they include alcohol.

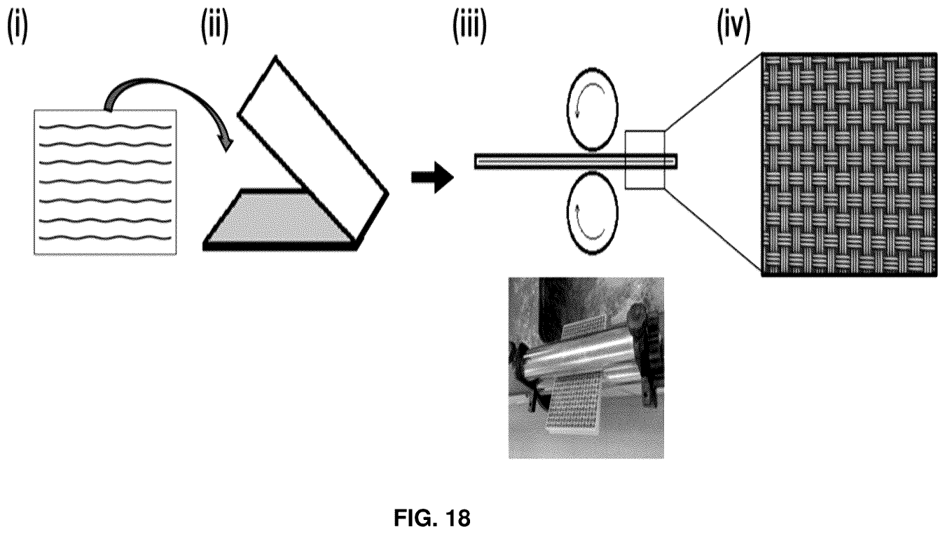

The wet wipes may also be made using a rolling press, such as depicted in FIG. 18. Advantageously, a rolling press may also be used to emboss the wet wipes with patterns.

Without wishing to be bound by theory, it is believed that it may be possible to improve the tactile properties of the wipes through compression. Therefore, methods for making the wet wipes may further include one or more compression steps, including at elevated temperatures.

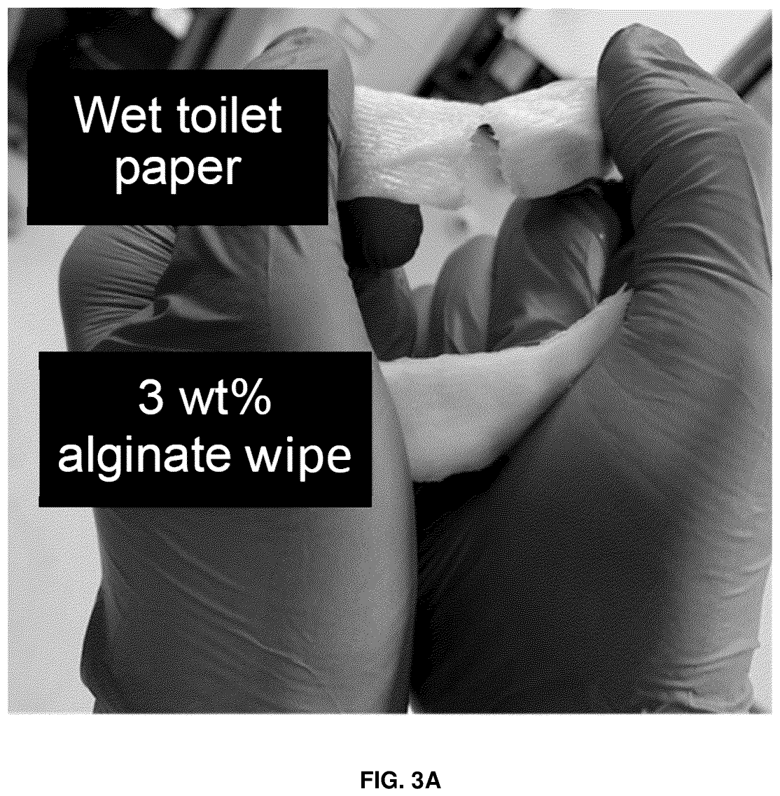

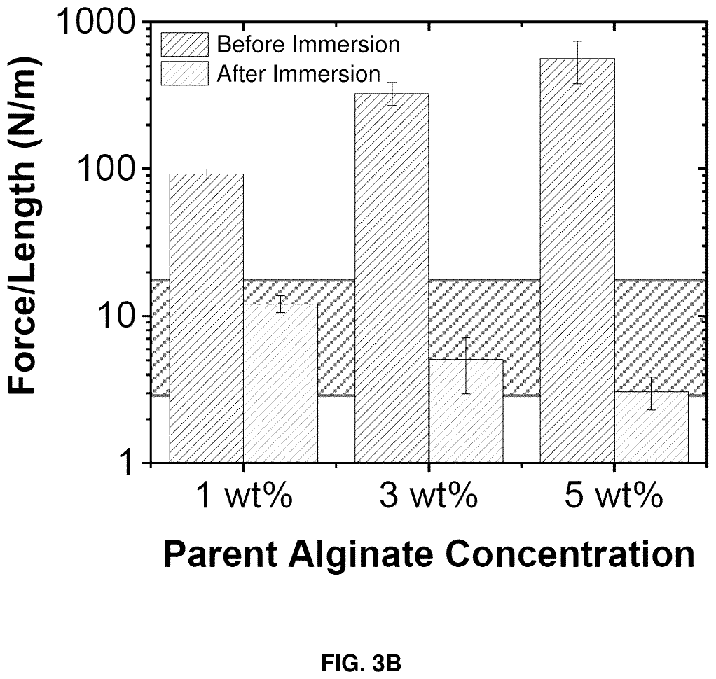

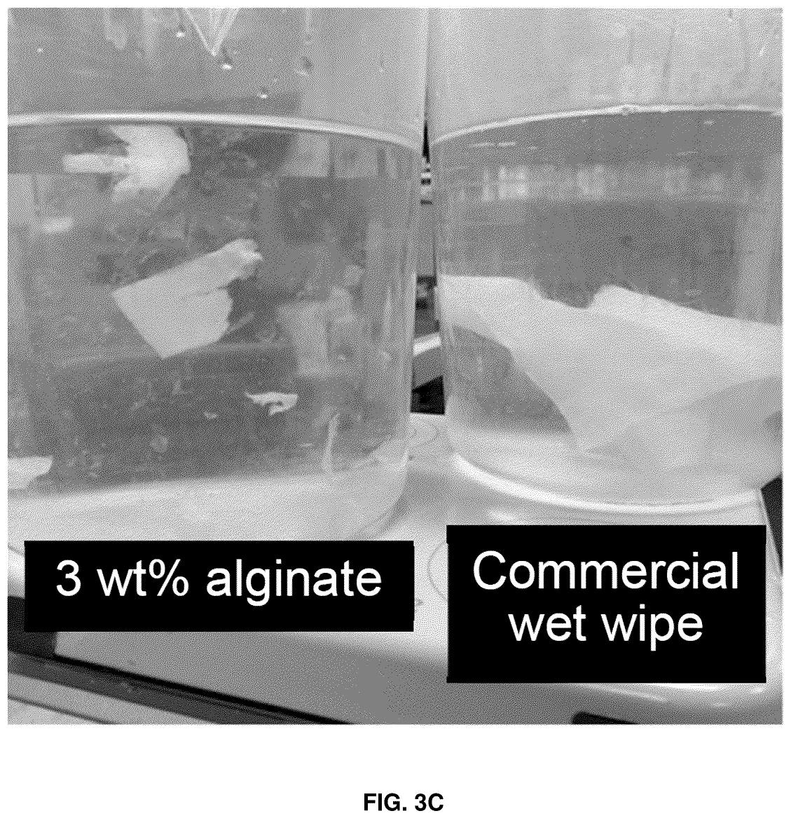

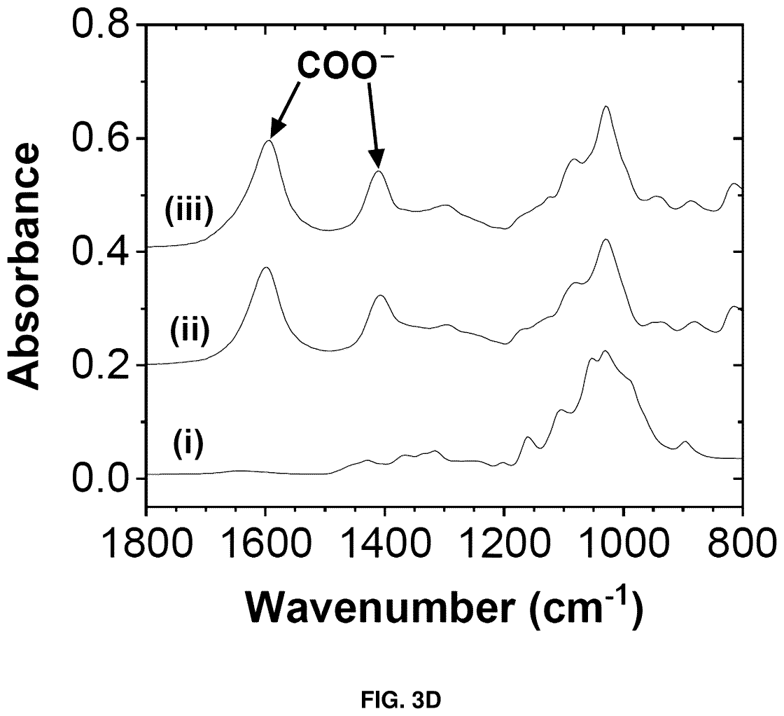

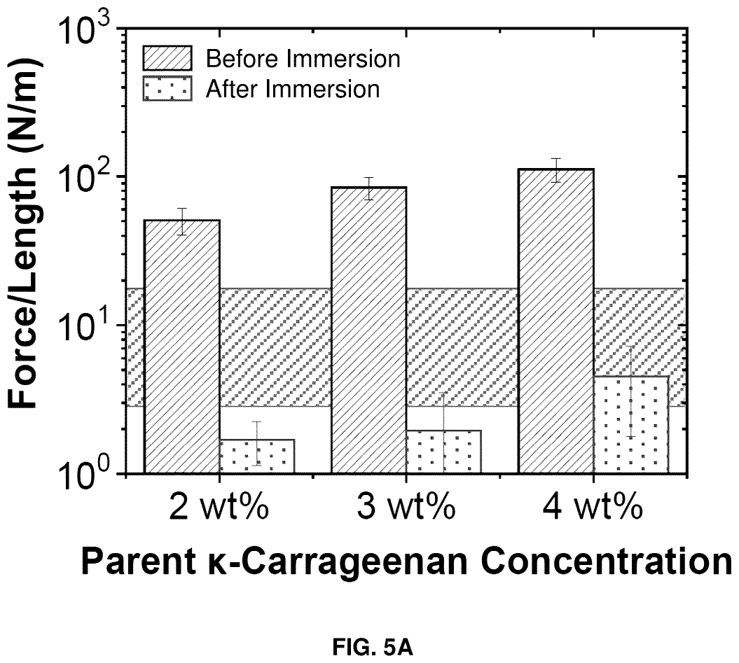

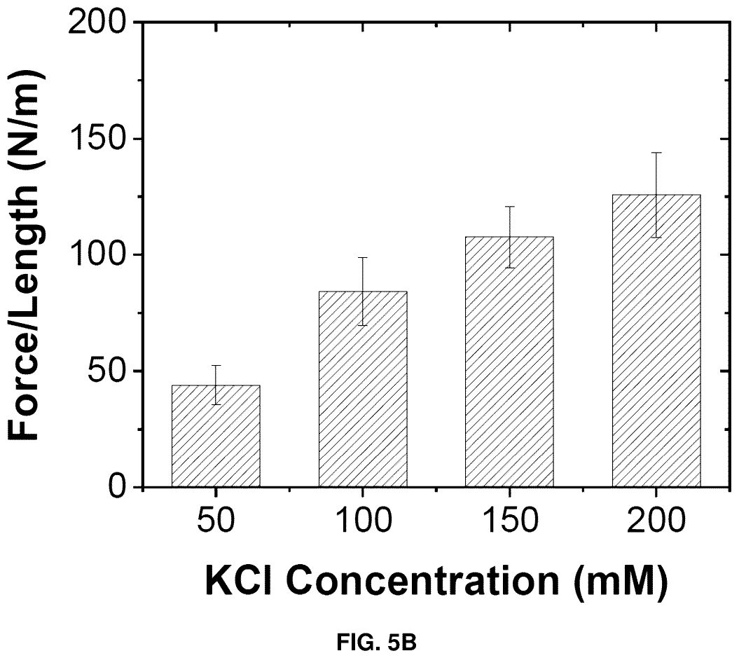

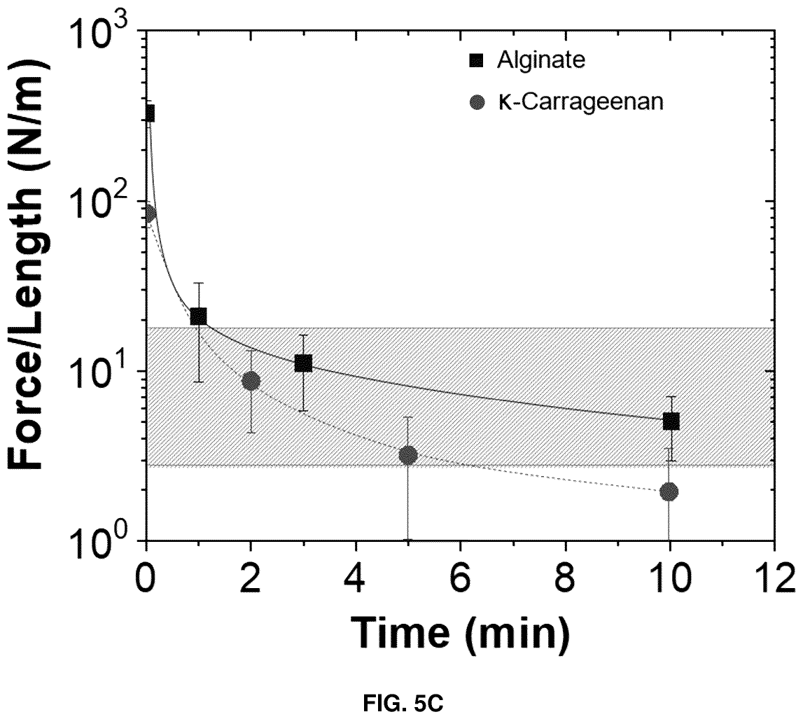

In both non-limiting examples, using either carrageenan or alginate, the wet wipes provide sufficient strength and turn into wet toilet paper upon simulated flushing (FIGS. 3B, 5A). The tensile strengths are comparable to those of commercial “flushable” wet wipes, which (based on a study of various commercial products) have been reported to have an average strength of 154±52 N/m. The flushable wet wipes described herein have a tensile strength during use of about 100 N/m. Upon simulated flushing (i.e., within minutes of immersion in excess water), the wipe tensile strength drops to that of wet toilet paper, or even less than that of wet toilet paper. Upon flushing, the wet wipes clear the drain line and pass PAS 3 slosh box testing, which is a type of testing used to assess the potential for a product to disintegrate when subjected to mechanical agitation in water. The eluted biopolymer (assuming a 20× dilution in the wastewater collection system) does not harm biological wastewater treatment.

As seen in FIG. 5B, aqueous, kappa-carrageenan-based wet wipes can also be strengthened by raising the monovalent salt (e.g., KCl) content. The wet wipes exemplified in FIG. 5B were prepared from 3 wt % kappa-carrageenan and 2 toilet paper layers. Increasing concentration of KCl leads to stronger wipes.

Advantageously, in some embodiments, the wet wipes are truly flushable because they rapidly disperse upon flushing, and leave behind only wet toilet paper debris and a non-toxic natural polysaccharide as its degradation products. The wet wipes can also be microplastics-free, alcohol-free, and made from widely available biobased feedstocks, making the wet wipes environmentally friendly. This addresses environmental threats caused by microplastics, and the risks to wipe producers due to stricter emerging/future environmental regulations. Additionally, the materials involved in making the wet wipes described herein are inexpensive and readily available.

Furthermore, the structure of the wet wipes is convenient and easy to use. The individual sheets can be packaged in a resealable container or pouch, which helps to keep the wipes moist and prevents them from drying out. The wet wipes can be easily accessed and used as needed, and then disposed of in a trash can, other waste receptacle, or flushed in a toilet without concern of clogging pipes or introducing microplastics into the environment.

The wet wipes described herein may also be made available via a kit containing one or more key components. A non-limiting example of such a kit comprises a water-dispersible substrate material and components of a biopolymer composition in separate containers, where the containers may or may not be present in a combined configuration. For example, a first container may include a plurality of sheets of toilet paper, and a second container may include a carrageenan or alginate, a monovalent salt, and water. Many other kits are possible and encompassed within the scope of the present disclosure. The kits may further include instructions for using the components of the kit to prepare wet wipes. The instructions may be recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded.

### EXAMPLES

These examples demonstrate the transformation of toilet paper into wet wipes that turn back into (readily dispersible) toilet paper upon being flushed, by interspersing toilet paper with stimulus-responsive biopolymeric gels. To achieve this, three types of biopolymeric gels were evaluated: (1) gels formed through surfactant/cationic biopolymer complexation; (2) alcohol-based gels formed through the ethanol-triggered phase inversion of the nontoxic biopolymer alginate; and (3) hydrogels based on kappa-carrageenan. It was discovered that the surfactant/cationic biopolymer networks did not produce the desired effect. However, the generation of wet, toilet paper-based tissues with mechanical strengths comparable to commercial flushable wet wipes whose properties revert to those of wet toilet paper upon simulated flushing is demonstrated in these examples using both aqueous and ethanolic (i.e., alcohol) wipes. The impacts of compositional parameters on the wipe mechanical strength, and loss of this mechanical strength upon simulated flushing, were analyzed by tensile mechanical testing. Moreover, the elution of the biopolymers from these wet wipes upon simulated flushing was analyzed by Fourier-transform infrared spectroscopy (FTIR) and confocal laser-scanning microscopy (CLSM). To link the wet wipe properties to those of their constituent gels, the biopolymer gel rheology was also probed. Furthermore, to test flushability in a more realistic setting, best-performing prototypes were sent for further testing at the Toronto Metropolitan University (to confirm that the wipes indeed cleared the toilet and drain line and, thereafter, dispersed).

Besides establishing the flushability of the wipes, the impact of their degradation products on wastewater treatment was also investigated. To this end, batch bioreactors were constructed and operated with raw wastewater and biomass from a local wastewater treatment plant. Using the bioreactors, the changes in the performance of the wastewater treatment process were monitored, as well as the quantity and activity of biomass (a.k.a., activated sludge) in the presence of wet-wipes and their eluted chemicals (alginate, ethanol, kappa-carrageenan, and KCl). The bioreactor performance was determined by measuring their sludge volume indeces (SVIs) and removal of chemical oxygen demand (COD). The SVI indicates the settleability of biomass and is a commonly monitored parameter in determining the bioreactor performance of the activated sludge process, while the COD is determined to estimate the amount of pollutants in wastewater. Further, the mixed liquor suspended solids (MLSS) and mixed liquor volatile suspended solids (MLVSS) were determined for total biomass quantification, and the active biomass quantities were ascertained using adenosine triphosphate (ATP) kits for wastewater biomass. Biomass stress indices (BSIs) in the presence of dissolved/dispersed wet wipes were also estimated from the obtained ATP results.

### Materials and Methods

All experiments were performed using deionized water with an 18.2 MΩ-cm resistivity from a Millipore Direct-Q 3 water purification system. Sodium alginate (from Macrocystis pyrifera; Mᵥ≈100-200 kDa, based on capillary viscometry, M:G≈60:40) was purchased from MP Biomedicals (Solon, OH), while the kappa-carrageenan was obtained from Sigma-Aldrich (St. Louis, MO). Ethanol and potassium chloride (KCl) were purchased from Decon Labs (King of Prussia, PA) and LabChem (Zelienople, PA), respectively. Fluoresceinamine, isomer I was bought from Fisher Scientific (Waltham, MA), and 4-(4,6-Dimethoxy-1,3,5-triazin-2-yl)-4-methylmorpholinium chloride (DMTMM) was obtained from TCI (Tokyo, Japan).

### Wet Wipe Preparation

Wet wipe samples were prepared from roughly 3.0 cm×5.5 cm toilet paper (Charmin Ultra Plus 2-ply) strips, prepared by cutting strips from toilet paper squares with scissors. To prepare alcohol wipes, 5 mL of 1-5 wt % alginate solution was poured onto the toilet paper strips, which were stacked into 1-3 layers. After allowing the alginate solutions to absorb for 5 min (which ensured thorough wetting of all toilet paper layers), 10 mL of 95 vol % ethanol was gently poured over the alginate solution-infused paper strips in petri dishes (Ø=9.4 cm). Upon gelling the alginate for about 15 min, the strips were inverted and allowed to gel for another 15 min. To also generate aqueous wet wipes, 5 mL of 2-4 wt % kappa-carrageenan solution at 70° C. was infused into toilet paper strips on a ˜50° C. petri dish (Ø=9.4 cm), which was kept on the heating plate to reach this temperature. To achieve this (and prevent the toilet paper from sticking to the glass petri dish), a TruOffice transparency film piece was first cut out and adhered to a petri dish surface with 3M Permanent Double-Sided Scotch® tape. The toilet paper strips were then placed on top of this transparency film and allowed to reach ˜50° C. After pouring approximately 5 mL of the hot kappa-carrageenan solution on the toilet paper and removing the excess hot kappa-carrageenan solution from the paper surface (following a 1-min infusion time) with a Kimwipe™, the kappa-carrageenan solution-infused wipes were immersed in room-temperature 50-200 mM KCl solutions for 5 min to generate kappa-carrageenan gels within the toilet paper matrices. Finally, to enhance their liquid water content and tactile properties, the wipes were perforated with a metallic brush (HERTZKO Self-Cleaning Slicker Brush) by gently pressing the wet wipes (kept on a hard plastic surface) with the brush during their infusion step in KCl, which produced small, water-filled holes.

To scale these samples up to a size that is more comparable to commercial wipes, the toilet paper squares were overlaid crosswise and trimmed to 9 cm×9 cm dimensions and, after being placed in a larger petri dish (Ø=14 cm), infused with 6-7 mL of alginate or kappa-carrageenan solution, which was spread using a nitrile glove-covered index finger by pressing the paper layers until the liquid was uniformly distributed throughout the toilet paper and the toilet paper surface was both flat and smooth. The alginate and kappa-carrageenan-based wipes were then immersed in ˜6-7 mL of either ethanol or aqueous KCl solution to gel the mixtures and perforated with the metallic, using the immersion times and perforation procedures described previously.

### Wet Wipe Characterization

The tensile strengths of the wipes were estimated from the maximum tensile force required to break the wipes. To this end, toilet paper was cut into 30-mm-wide strips and the wet wipes were prepared above. They were then loaded onto an Instron 5566 Universal Testing Machine (UTM; Norwood, MA) and pulled apart at a crosshead speed of 51 mm/min. The measurement was stopped once the wipes tore, and the tensile strength was calculated by dividing the maximum load sustained by the wipe width. Six replicate measurements were performed for each experimental condition.

Besides their mechanical characterization, the wipes were also imaged by digital photography and CLSM. To perform the fluorescence-based confocal imaging, the alginate was fluorescently labeled by fluoresceinamine, whereupon they were analyzed using a Leica TCS SP5 Multiphoton Laser Scanning Confocal Microscope (Wetzler, Germany) using both fluorescence and surface topography imaging.

### Dynamic Rheology

Additional analysis of the polyelectrolyte gel properties underlying the wet wipe mechanical strength was performed through dynamic rheology. To probe the rheology of alginate gels, 30 mL alginate solutions (1, 3 or 5 wt %) were first prepared. They were then poured into 600 mL, 0=8 cm beakers, submerged in 100 mL of ethanol, and allowed to gel for 1 h. The gels were then cut into centimeter-sized pieces and loaded onto a TA Instruments DHR-3 (New Castle, DE) stress-controlled rheometer equipped with 8-mm parallel plates. To also analyze the rheological properties of kappa-carrageenan gels, precursor solutions for their formation were first prepared by dissolving the kappa-carrageenan in 70° C. deionized water and adding stock solutions of KCl to obtain aqueous solutions of 2-4 wt % kappa-carrageenan and 0-200 mM KCl. These solutions were then poured onto 1-mL chambered microscope slides and gelled in a 25° C. water bath for 5 min, whereupon the gels were loaded onto the same TA Instruments DHR-3 rheometer equipped with 20-mm parallel plates.

After compressing the gels to a thickness of 0.5 mm, their rheological properties were analyzed after conditioning the samples at 15° C. (for the thermogelling kappa-carrageenan gels) and 25° C. (for the solvent-sensitive alginate gels) for 5 min. The linear viscoelastic region (where the storage and loss moduli were strain rate-independent) was first determined via strain amplitude sweep measurements, performed at 1, 10, and 100 rad/s angular frequencies and 0.1-10% strain rates. Upon establishing the linear viscoelastic regions, frequency sweep (1-100 rad/s) measurements were then performed on the ethanolic alginate gels at 0.1% strain amplitude. Conversely, for the temperature-sensitive kappa-carrageenan gels, temperature sweeps (15-80° C.) were conducted at a 1° C./min ramp rate, 1 rad/s frequency, and 0.1% strain amplitude, which enabled the comparison of both room-temperature gel strengths and thermal stabilities of the different kappa-carrageenan/KCl compositions. To minimize solvent loss during the rheological characterization, all samples (both ethanolic and aqueous) were covered with a solvent trap, and all measurements were performed in triplicate.

### Fluorescent Labeling of Alginate

Fluoresceinamine-labeled sodium alginate was synthesized by first preparing a 150 mL solution of 1 wt % alginate in 1×PBS. Upon dissolving the alginate, 0.038 g of DMTMM was added, which corresponded to a 0.02:1 DMTMM:alginate carboxylate group molar ratio. After mixing for 2 h, 0.048 g of fluoresceinamine (which corresponded to a 0.02:1 fluoresceinamine:alginate carboxylate group molar ratio) was added to the alginate/DMTMM solution and allowed to react with the DMTMM-activated alginate in the dark (and at room temperature) for 24 h. The fluorescently labeled alginate was then precipitated with 350 mL of methanol and, after chopping the precipitate into mm-sized pieces, washed with 500 mL of fresh methanol overnight. After removing the supernatant by vacuum filtration, the precipitate was centrifuged twice at 4000 rpm (3,220 g; for 1 h each time) in ˜30 mL methanol (using fresh methanol for each cycle) to further remove any unreacted fluoresceinamine using a 5810R Eppendorf centrifuge. Finally, the purified fluoresceinamine-labeled alginate was lyophilized overnight using a Labconco Freezone Plus 6-L lyophilizer to obtain a dry powder. This fluorescent labeling of alginate enabled the CLSM-based determination of whether the alginate persists within the toilet paper matrix upon the immersion of the alginate-based wet wipes in excess water.

### Simulated Flushing

As a crude initial model of simulated flushing (i.e., exposure to an excess of water), the wet wipes were dropped in 1 gal of tap water and agitated (inside a 4-L beaker) at either 200 rpm or 300 rpm using a 63 mm×10 mm magnetic stir bar at either room temperature or (to also explore their dissociation in cold and hot environments) 7° C. or 40° C. The above stirring speed was selected to represent a “worst-case scenario” for wipe dispersion (where agitation was a lot gentler than that during the flushing of a real toilet), so that, under normal flushing conditions, wipe weakening/degradation would occur even faster. After allowing the wipes to dissociate for variable (2-10 min) amounts of time, the wipes were carefully lifted from the water and stored in Ziploc® bags in a humidified chamber (70-80% RH) for further analysis. This further analysis included tensile strength testing (to determine the impact of simulated flushing on the wipe strength as a proxy for dispersibility), and CLSM (to ascertain the fate of the gelling biopolymer). To further characterize the origins of changes in the wipe mechanical properties, alginate and kappa-carrageenan presence on the flushed wipe surfaces was checked by Varian Excalibur Series Fourier Transform Infrared Spectroscopy (FTIR). After either 30 min (for alginate-based wipes) or 10 min (for kappa-carrageenan wipes) of simulated flushing, the wipes were dried overnight in a 40° C. oven, after which the attenuated total reflection (ATR) method of FTIR was used to analyze the wipe surface. A background spectrum was measured, followed by gently touching the wipe surface with a germanium crystal to obtain the ATR spectra. The absorbance peaks were then compared to the literature spectra to identify the presence or absence of alginate or kappa-carrageenan. Each FTIR measurement was performed using two replicate samples.

### Results and Discussion

Interspersing toilet paper with stimulus-responsive biopolymeric gels can transform the toilet paper into wet wipes that turn back into (readily dispersible) toilet paper upon being flushed. To achieve this outcome, three types of biopolymeric gels were examined: two that produced the desired flushability effect and one that did not. The first investigated approach to generating the wet wipes was by forming surfactant/cationic biopolymer complexes within the toilet paper, which would dissociate upon flushing. The cationic biopolymers were both quaternized polysaccharides (quaternized hydroxyethyl cellulose and quaternized chitosan), while the surfactants were fatty acid salts (all of which are molecules already used in cosmetic, personal care, and biomedical applications). The surfactant/biopolymer complexes successfully formed and dissociated on demand upon being immersed/agitated in an excess of water (as would occur upon flushing). However, these complexes did not provide the required strengthening effect. To overcome this challenge, two other biopolymer-based approaches were evaluated: one that requires the use of ethanol as a solvent (which resulted in the formation of alcohol wipes that turn into toilet paper when flushed), and another that works with water-based (i.e., aqueous) wet wipes.

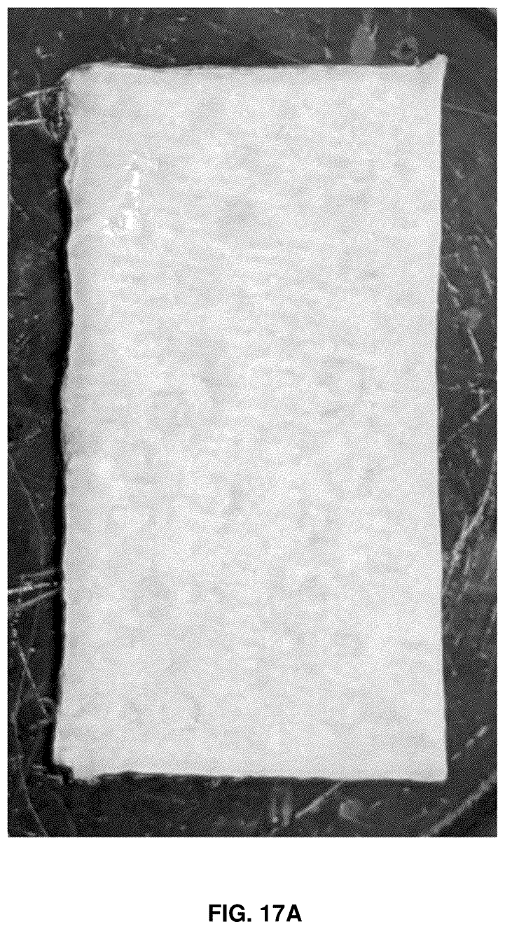

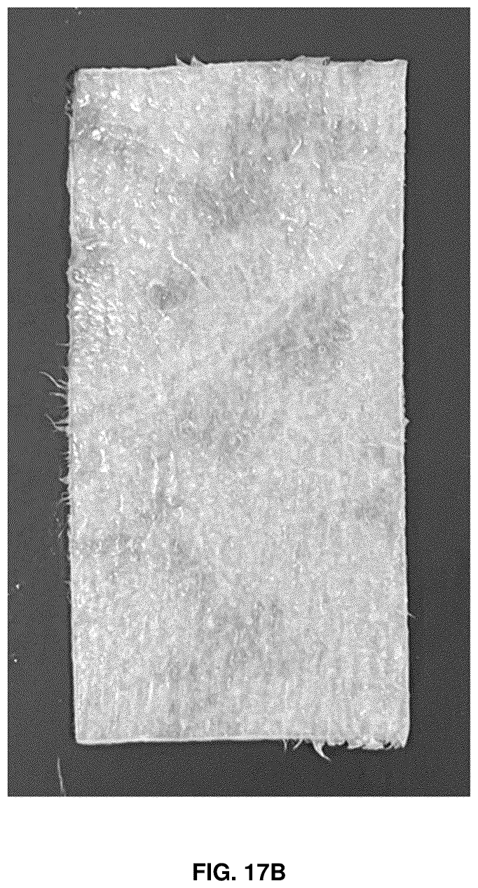

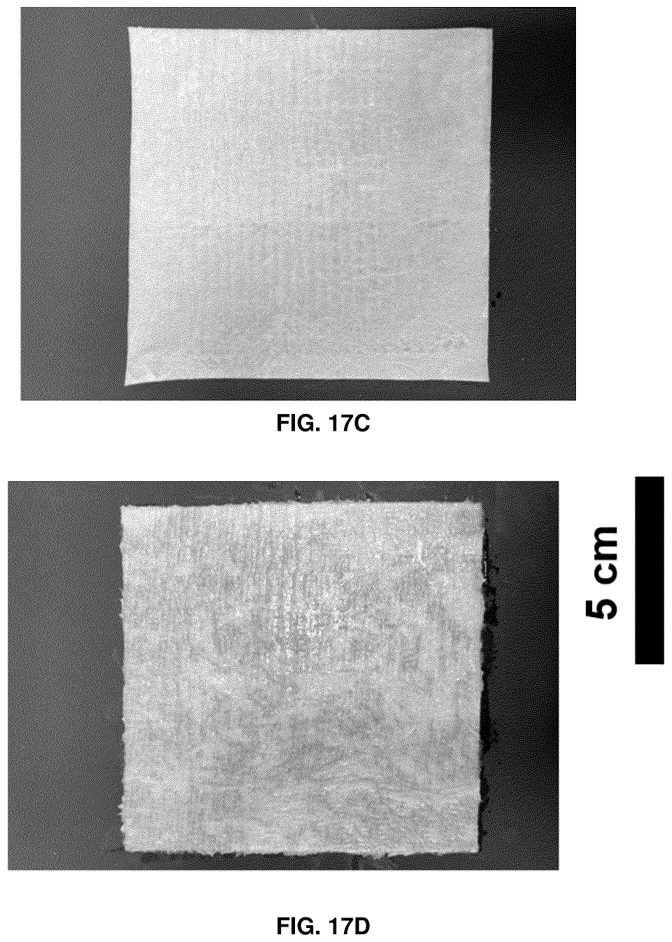

After infusing of toilet papers with the biopolymer (alginate and kappa-carrageenan) solutions, and gelling the biopolymers, alcohol or water-based wet wipes were obtained (FIGS. 2A-2B, 17A-17D). When alginate solution was exposed to ethanol (which, besides its disinfecting properties, acted as an antisolvent), the solution underwent phase inversion and formed an ethanolic gel phase that (when multiple toilet paper layers were used) adhered the layers together into mechanically reinforced wipes. Conversely, the kappa-carrageenan gelation was caused by a well-known cooling-induced transition from the coil-like to helical polymer configuration, which led to the inter- and intramolecular hydrogen bond-driven aggregation and gelation of the kappa-carrageenan chains. This coil-to-helix transition and, consequently, paper-strengthening gelation was further enhanced by KCl, which enabled the wipe strengths to also be tailored by varying the KCl concentration.

### Alcohol Wipe Properties

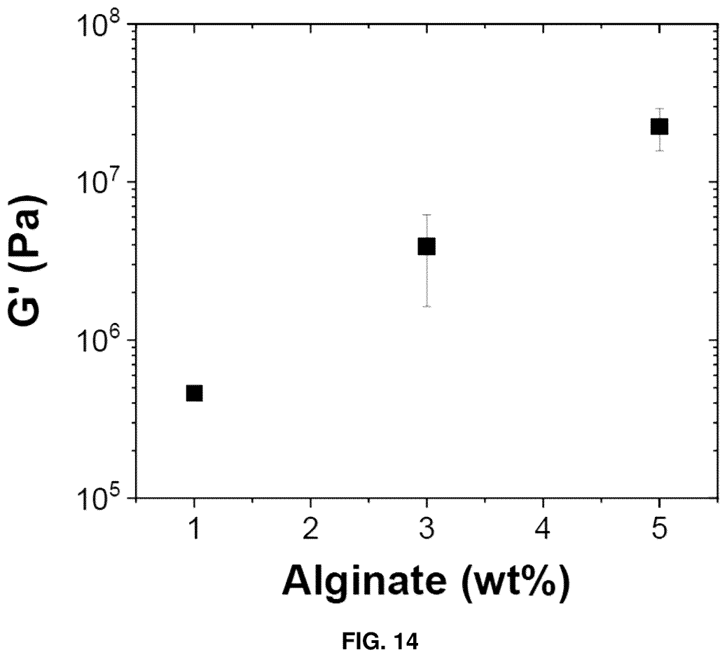

The ethanol-based approach uses the non-toxic and widely available polysaccharide alginate, which is water-soluble but forms strong gels when the water is replaced with alcohols. Thus, infusion of toilet paper (stacks of three toilet paper sheets) with aqueous alginate solution followed by its immersion in ethanol forms robust ethanol-based wet wipes, whose mechanical strength can be tailored by varying the alginate concentrations in the aqueous precursor solutions used to form the wipes (see FIGS. 3A-3B). While wet stacks of toilet paper were easy to tear, the interspersion of the ethanolic alginate gel within the toilet paper multilayers made them significantly stronger (FIG. 3A). Moreover, as the alginate concentration in the precursor solution was increased, the tensile strengths of the wipes (normalized to the wipe width) increased from approximately 100 N/m for 1 wt % alginate to roughly 500 N/m for 5 wt % alginate (FIG. 3B). Notably, each of these alginate gel-infused wipes had tensile strengths either near or above the target ˜100 N/m wipe strength, with the strengths of wet wipes prepared from 3 and 5 wt % alginate solutions comparing favorably to the typical 154±52 N/m (mean±SD) tensile strengths of commercial “flushable” wet wipes (estimated by averaging the published strengths of various commercially available wet wipe products). The stronger gel networks obtained at higher alginate concentrations were qualitatively consistent with both prior analyses of alginate gels and the increased stiffness of the more concentrated alginate alcogels revealed by dynamic rheology (FIG. 14).

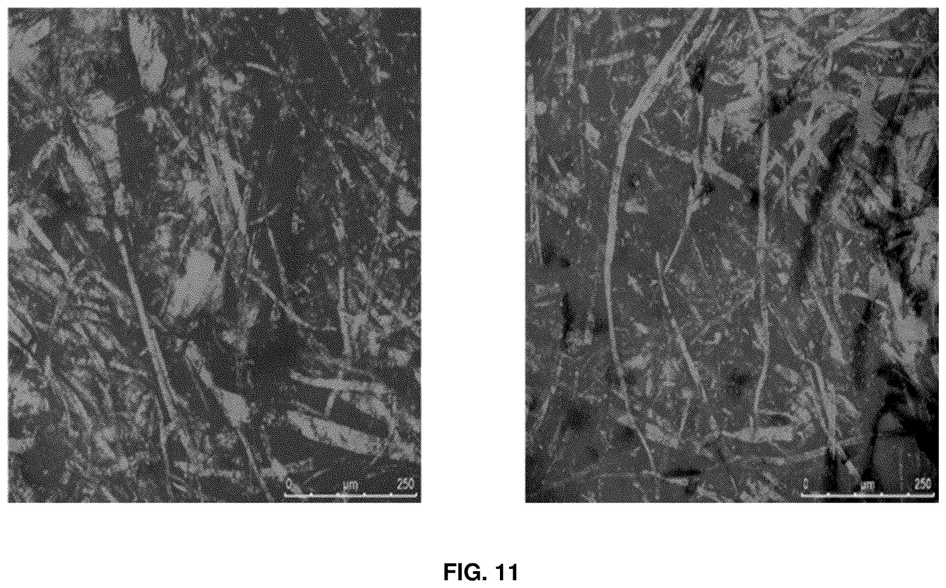

When the wipes are placed in an excess of water (such as would occur upon flushing), however, the ethanol diffused out and the alginate gels dissolved. This dissolution significantly weakens the wipes, and their strength on flushing becomes comparable to that of regular, three-layer stacks of toilet paper (as seen 10 min after immersion in 4 L of water in FIG. 3B). This simulated flushing-triggered weakening of the wipes made them readily dispersible. Indeed, the strength of the wipes upon such water immersion decreased at higher alginate contents, indicating that, once the gels dissolve, the presence of biopolymers such as alginate may facilitate wet wipe dispersion. Consequently, unlike conventional commercial wet wipes, the wipes readily dispersed when agitated in an excess of water (FIG. 3C). In fact, as the alginate concentration increased, the tensile strengths of the “flushed” wipes decreased even more. Further, to determine whether the dispersibility was caused by the elution of the biopolymer from the wipes, the wipes were analyzed by FTIR both before and after water immersion (FIG. 3D). This analysis, based on the persistence of the characteristic COO⁻ alginate peaks (which are absent from alginate-free toilet paper) after water immersion, revealed that the alginate does not yet diffuse from the toilet paper-based wipes when their tensile strength drops to that of the wet toilet paper. Indeed, even after 30 min of these alcohol-based wipes being immersed in water, the alginate was not completely eluted from these wipes. To confirm the absence of these peaks in the toilet paper, an additional control spectrum was obtained, which indicated the absence of COO⁻ peaks in alginate-free toilet paper. This FTIR analysis showed that the drop in wipe tensile strength upon simulated flushing likely reflected a weakening and disintegration of the gel network rather than removal of the biopolymer from the toilet paper matrix. This result was also confirmed by CLSM performed upon fluorescein-labeling of the alginate, which confirmed that the fluorescent alginate persisted within the toilet paper matrix (FIG. 11). The fluorescein-labeled alginate (indicated by the green background color in the images) was present in the space between the (orange) toilet paper fibers both before and 10 min after the simulated flushing. Thus, though the alcogel network degrades (due the replacement of the ethanol with the ambient water), the solubilized alginate molecules persist within the toilet paper matrix, which likely reflects the diffusivity of these polymer molecules being much slower than that of the rapidly exchanged, small-molecule (water and ethanol) solvents. The transformation of the wipes into regular, wet toilet paper was evidently caused by the dissolution of the gel into a toilet paper-entrapped alginate solution rather than the elution of the biopolymer from the toilet paper matrix. Disintegration of the network rather than biopolymer elution weakens the wet wipes. Since the alginate did not completely elute from the wipes even after 30 min of simulated flushing, this correlation between the drop in tensile wipe strength and their parent biopolymer content indicates that higher alginate concentrations actually aid wipe dispersibility.

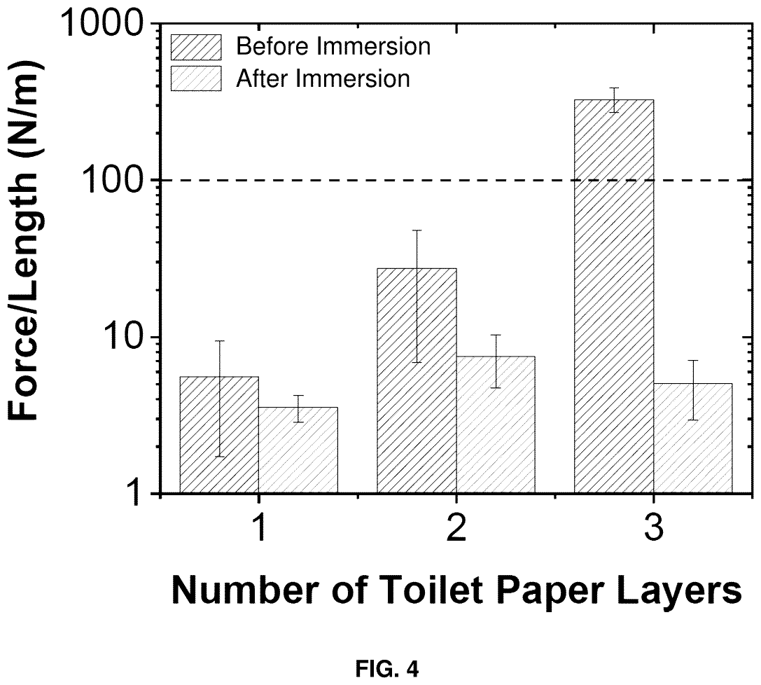

Besides varying the parent alginate solution concentration, the number of toilet paper layers used for the wipe preparation had a profound impact on the wipe properties (FIG. 4). It was determined that having three layers of alginate gel-infused toilet paper was important for the strong alginate-based alcohol wipe formation. When the 3 wt % alginate solution-based composition based on three layers was simplified to only include one or two layers of base toilet paper, the average tensile strength of the toilet paper dropped precipitously, from more than 300 N/m (which significantly exceeded the ˜100 N/m target strength) to ˜5-30 N/m range (FIG. 4). Besides being much too weak, the mechanical properties of these wipes proved to be highly variable (as seen by the much larger error bars). Thus, at least at the 3 wt % parent alginate solution concentration, three layers of toilet paper were needed to produce a strong flushable alcohol wipe.

### Aqueous Wet Wipe Properties

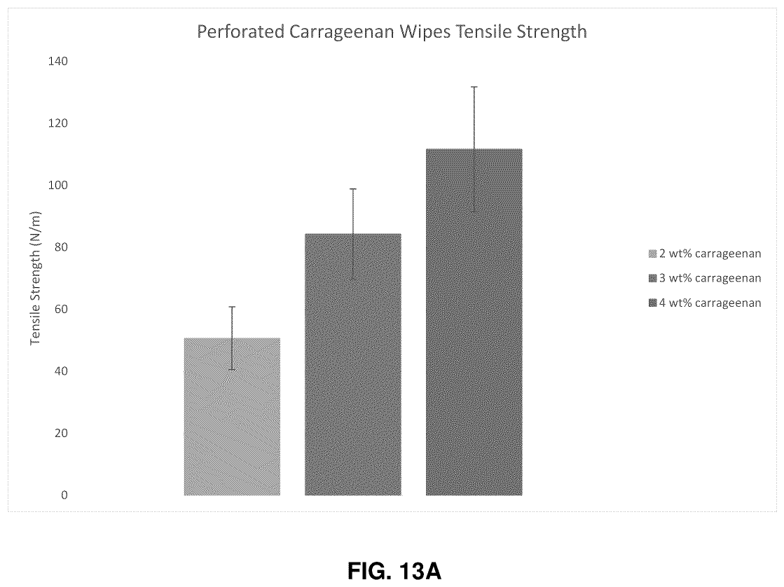

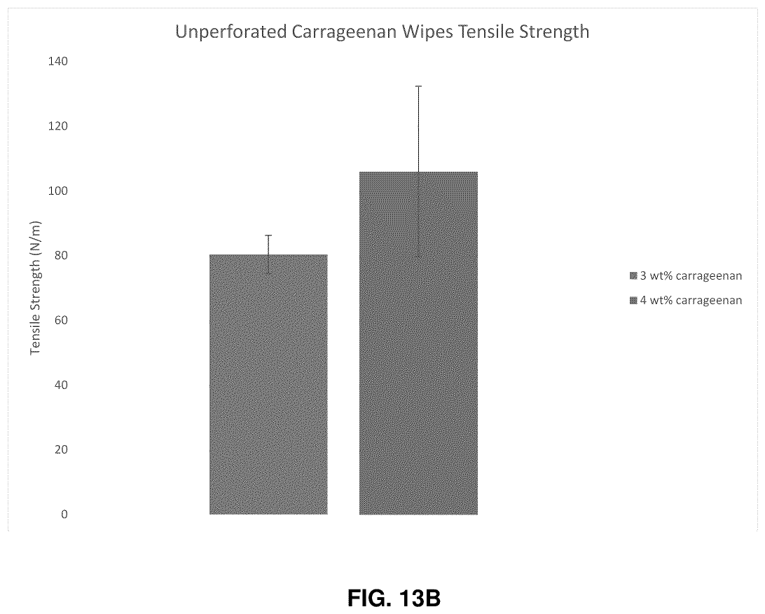

After exploring several alcohol-free biopolymer-based approaches to the wet wipes, it was also demonstrated that strong water-based wipes, which turn into wet toilet paper upon simulated flushing, can be formed by interspersing the toilet paper with hot kappa-carrageenan solutions (FIGS. 2A, 17B, 17D). These solutions (especially in the presence of KCl) form strong gels on cooling. Harnessing this effect, wipes were generated by placing the toilet paper on a ˜50° C. plate, infusing it with 70° C. 2-4 wt % kappa-carrageenan solutions, and then immersing the biopolymer solution-infused toilet paper into room-temperature 100 mM KCl solution to generate strong kappa-carrageenan gels within the toilet paper matrix. Finally, to increase their liquid (rather than gelled) water content, the wipes were perforated with a metallic brush, which produced small, water-filled holes. This perforation improved the tactile properties of the wipes without affecting their tensile strength (FIGS. 13A-13B). As in the case of the alginate wipes, the wipe strength increased with the parent kappa-carrageenan solution concentration (FIG. 5A) and with number of base toilet paper layers used. As seen in FIG. 5A and Table 1, there was a clear correlation between increased carrageenan concentration and increased tensile strength. The wet wipe with 4 wt % kappa-carrageenan exhibited the greatest tensile strength.

TABLE 1
Results of tensile strength testing of perforated
wet wipes formed using two toilet paper layers.

| | 2 wt % kappa-carrageenan | | 3 wt % kappa-carrageenan | | 4 wt % kappa-carrageenan | |
|---|---|---|---|---|---|---|
| | Force at | Tensile | Force at | Tensile | Force at | Tensile |
| | break (N) | strength (N/m) | break (N) | strength (N/m) | break (N) | strength (N/m) |
| | 1.49 | 49.6 | 1.75 | 58.2 | 2.53 | 84.4 |
| | 1.64 | 54.7 | 2.72 | 90.8 | 3.57 | 119.0 |
| | 1.97 | 65.6 | 2.46 | 82.1 | 4.08 | 136.0 |
| | 1.47 | 48.9 | 2.97 | 98.9 | 2.88 | 95.9 |
| | 1.02 | 34.0 | 2.42 | 80.7 | 3.91 | 130.2 |
| | 1.53 | 51.0 | 2.84 | 94.7 | 3.13 | 104.4 |
| Average: | | 50.6 | | 84.3 | | 111.6 |
| Std. Dev.: | | 10.2 | | 14.6 | | 20.2 |

The experiment was repeated for the 3 wt % and 4 wt % carrageenan wipes except without perforation (which was used to create solvent-filled pores within the wipes, increase their liquid water content, and improve their tactile properties). The results are shown in Table 2 and FIG. 13B and indicate that perforation, which enhances the liquid content and tactile properties of the wipes, does not affect their mechanical strength. Specifically, two-toilet paper-layer wipes prepared from 3 and 4 wt % kappa-carrageenan solutions without the perforations had average tensile strengths of 80±6 and 106±26 N/m (mean±SD), respectively. These values were similar to those obtained for the same toilet paper/kappa-carrageenan compositions with the perforations (84±15 and 112±20 N/m, respectively), and confirmed that the perforation procedure did not impact the tensile strength.

TABLE 2
Results of tensile strength testing of unperforated
wet wipes formed using two toilet paper layers.

| | 3 wt % kappa-carrageenan | | 4 wt % kappa-carrageenan | |
|---|---|---|---|---|
| | Force at | Tensile | Force at | Tensile |
| | break (N) | strength (N/m) | break (N) | strength (N/m) |
| | 2.35 | 78.2 | 3.21 | 106.9 |
| | 2.42 | 80.7 | 3.16 | 105.3 |
| | 2.48 | 82.7 | 1.73 | 57.6 |
| | 2.10 | 70.1 | 4.06 | 135.4 |
| | 2.64 | 87.9 | 3.25 | 108.3 |
| | 2.48 | 82.5 | 3.67 | 122.2 |
| Average: | | 80.4 | | 106.0 |
| Std. Dev.: | | 6.0 | | 26.3 |

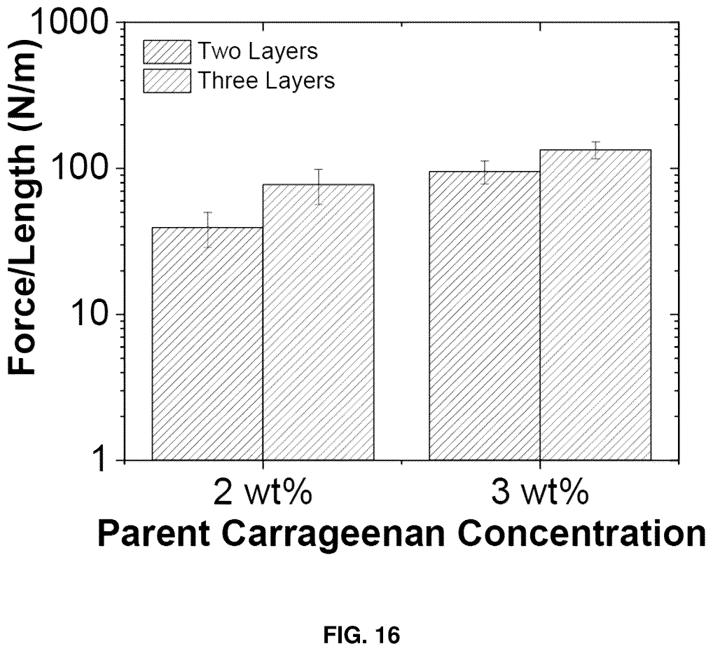

When higher kappa-carrageenan concentrations and multiple base toilet paper layers were used, the tensile strength of these wipes either reached or exceeded the ˜100 N/m target. When the wipes were prepared using two toilet paper layers and 100 mM KCl, the average tensile strength increased from 50 N/m to 111 N/m as the precursor kappa-carrageenan solution concentration was raised from 2 to 4 wt %. This increase was qualitatively consistent with prior experiments on the polymer concentration effects on the rheology of kappa-carrageenan gels, including those conducted as part of these examples. The impact of the kappa-carrageenan compositions on the tactile properties of the wipes was evident. When the wipes were prepared from the highest 4 wt % kappa-carrageenan solutions, they became inflexible, which (based on qualitative observation upon their manual handling) departed from the tactile feel of conventional wet wipes. The same happened when these wipes were prepared using three toilet paper layers instead of two. To limit this stiffening effect, the intermediate 3 wt % parent kappa-carrageenan concentration and two toilet paper layers were selected as an advantageous composition (despite the wipes prepared using 4 wt % kappa-carrageenan and three toilet paper layers being stronger; see FIGS. 5A, 16). The two-layered wipes prepared using 3 wt % kappa-carrageenan, with tensile strengths of 84±15 N/m (mean±SD), were therefore deemed to be the closest to the commercially advantageous wipe formulation. Though these tensile strengths of the 3 wt % kappa-carrageenan-based wet wipes were on the same (˜100 N/m) order as the 154±52 N/m figure reported for commercial flushable wet wipes, they were slightly weaker. To this end, whether the wipes can be strengthened by increasing the KCl concentration used in their preparation was evaluated (as KCl is a kappa-carrageenan gelation promoter and, thus, increases the kappa-carrageenan gel strength). As the KCl concentration used in the wipe preparation was raised from 50 to 200 mM, the tensile strength of kappa-carrageenan wipes monotonically increased (FIG. 5B). This result was consistent with the rheological analysis of the kappa-carrageenan gels used herein. Thus, the tensile strengths could be tuned not only by changing the kappa-carrageenan concentration but also by varying the KCl contents. Further, it may be possible to exceed the target tensile wipe strength while preserving wipe-like tactile properties, such as by utilizing the 3.0-3.5 wt % kappa-carrageenan concentration range. Once these kappa-carrageenan-based wipes are placed in excess water (as when they are flushed), the KCl—which serves as the gelation promoter—rapidly diffuses out, which weakens and/or dissolves the gel. The wet wipes therefore again become much weaker, with mechanical strengths akin to that of wet toilet paper (FIG. 5A).

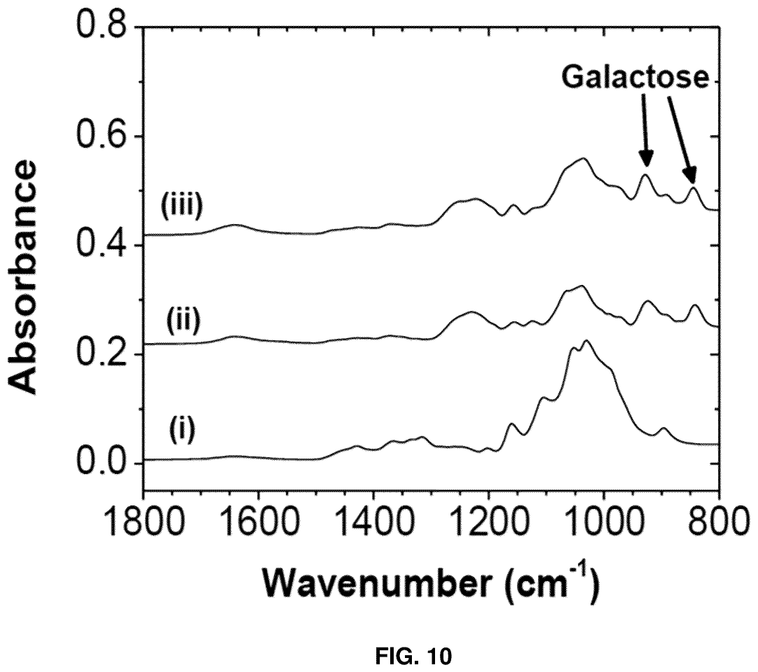

Since the potential of these wet wipes to clog wastewater collection systems decreases as their degradation and dispersion are accelerated, the rate at which the tensile strengths of both alginate/ethanol and kappa-carrageenan/water-based wipes decay upon their immersion in 4 L of water (FIG. 5C) was also characterized. The carrageenan-containing wet wipes lose almost all tensile strength within a few minutes upon immersion in water, demonstrating flushability. For both wipe types, the average wipe strengths dropped to levels comparable with those of wet toilet paper within 2-3 min. As seen with the alginate alcogels, the degradation of the kappa-carrageenan gel network rather than elution of the biopolymer itself caused the loss of tensile strength, which was evident from FTIR analysis (FIG. 10). As seen in FIG. 10, the characteristic peak for slowly diffusing kappa-carrageenan persist even after simulated flushing. Ten min after their water immersion, FTIR spectroscopy revealed the presence of the galactose groups of kappa-carrageenan within the “flushed” wipes, which were absent from the base toilet paper. The wipe-weakening gel degradation was likely either caused by a simple water uptake by/dilution of the kappa-carrageenan or elution of monovalent potassium ions into the surrounding tap water. Irrespective of its mechanism, however, this reduction of tensile strengths of the wipes (prepared at all investigated kappa-carrageenan concentrations) indicates the ability of these hydrogel/wet toilet paper composites to be used as flushable wipes. Thus, both the alcohol and water-based wet wipe embodiments achieve the objective of rapidly turning into regular wet toilet paper when placed in excess water (at least in terms of their mechanical behavior). Importantly, this effect is achieved over the full (7-40° C.) range of temperatures examined in this example, which indicates that this could prevent the buildup of wipes in wastewater collection and treatment systems regardless of whether the wastewater collection systems are cold or warm. The flushability of the 3 wt % alginate and three-toilet-paper-layer alcohol wipe and the 3 wt % kappa-carrageenan and two-toilet-paper-layer alcohol-free wipe embodiment (which was indicated by the data in FIGS. 3-5) was also confirmed through drain line clearance and PAS3 slosh box testing at the Toronto Metropolitan University, which further showed that the strength and flushability of the wet wipes are advantageous.

Interestingly, following their water immersion, the tensile strength of the 4 wt % kappa-carrageenan wipes remained higher than those of the 2 and 3 wt % kappa-carrageenan wipes (FIG. 5A). This trend opposed that obtained for the alginate wipes where the tensile strengths of the wipes after water immersion, in contrast, dropped with the increasing alginate concentration (FIG. 3B). This reflected either (1) a greater tendency of dissolved alginate in the flushed wipe to aid fiber dispersion compared to kappa-carrageenan, or (2) a less thorough dissociation of kappa-carrageenan gels upon the water immersion compared to alginate.

### Kinetic Analysis of Flushability

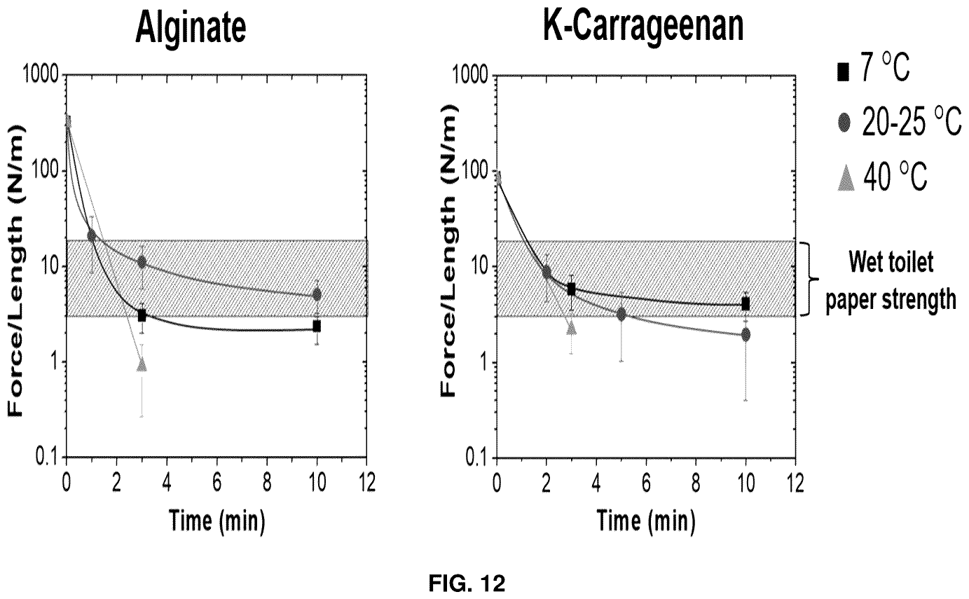

Since the potential of the wet wipes to clog wastewater collection and treatment systems decreases as their degradation and dispersion are accelerated, the rates at which the tensile strengths of both alginate alcogel and kappa-carrageenan hydrogel-based wipes decay upon their simulated flushing were evaluated (FIG. 12). This analysis revealed that, within 2-3 min of simulated flushing, the average tensile strengths of these wipes decrease to values comparable to that of three layers of wet toilet paper (FIG. 12). This rapid weakening occurred irrespective of the wipe composition used (alcohol-based or water-based) and demonstrated that wipe dispersal could be achieved relatively quickly.

Importantly, the rapid conversion of the wipes into wet toilet paper can be achieved in both cold (7° C.) and warm (40° C.) water, which indicates these flushable wipe designs can be used in both cold and hot climates. For the alginate-based wet wipes, however, the tensile strength reduction occurred slightly faster at 7° C. than at 25° C. A possible explanation for this effect could be that, at 7° C., rates of alginate elution from the wipes should be marginally slower than at 25° C. This modest difference in diffusion rates could increase the amount of dissociated alginate persisting in the toilet paper at 7° C. which (as shown in FIG. 3B) can weaken the toilet paper upon flushing and aid wipe dispersibility. When the wipes were placed in warmer, 40° C. water, however, their tensile strengths dropped sharply, and after 10 min of simulated flushing, the wet toilet paper they left behind completely broke apart (FIG. 12, left).

Unlike the alginate alcogel-based wipes, the rate of the thermogelling kappa-carrageenan-based wipe degradation increased monotonically with temperature, with the wet toilet paper again breaking apart by the 10-min time point (FIG. 12, right). This monotonic trend likely reflected (1) higher temperatures reducing the stability of the thermogelling kappa-carrageenan hydrogels, and (2) the presence of dissociated kappa-carrageenan—which may persist within the toilet paper longer at lower temperatures—not appearing to weaken the toilet paper matrix. Taken together, these results indicate that the wet wipes (across the temperature range studied here) degrade quickly, which will prevent the buildup of wipes in wastewater collection and treatment systems.

### Rheological Analysis

Compositional effects on the rheological properties of alginate alcogels were characterized through dynamic rheology. Though the gelation in ethanol likely produced inhomogeneous gels, and this inhomogeneity may have been further exacerbated by the breaking of these gels into pieces before their loading into the rheometer, there was a clear increase in their stiffness with the concentration of their parent aqueous alginate solutions. As this concentration was raised from 1 to 5 wt %, the apparent storage moduli (G′) measured by dynamic rheology (at to =1 rad/s) increased sharply (FIG. 14). Though there is uncertainty in the quantitative accuracy of these values, the trend obtained through these measurements provides strong evidence for the strengthening of the gel at higher alginate concentrations (which likely reflects an increased concentration of interpolymer linkages).

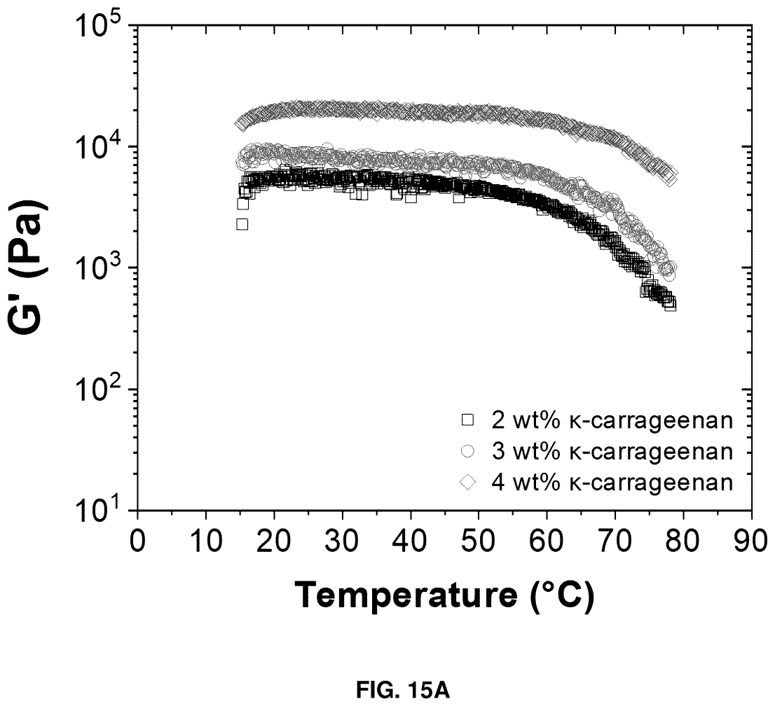

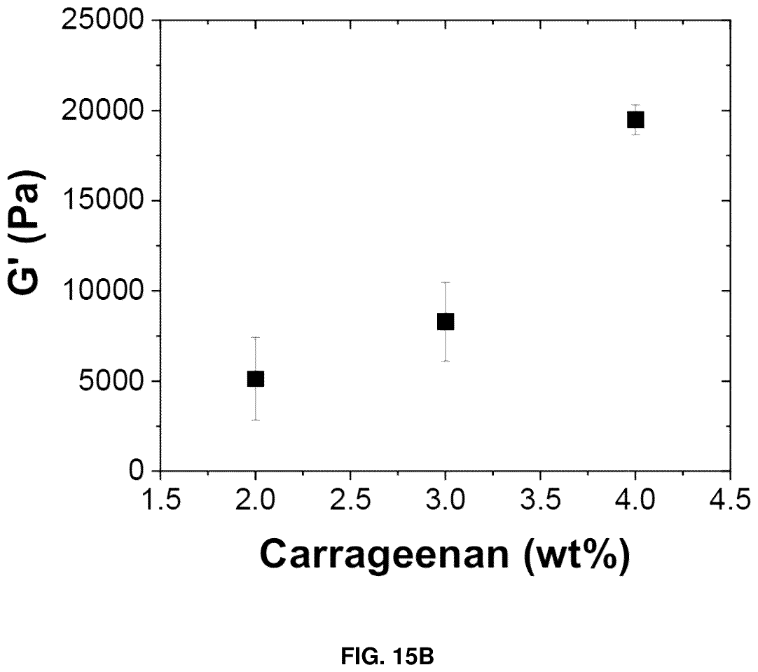

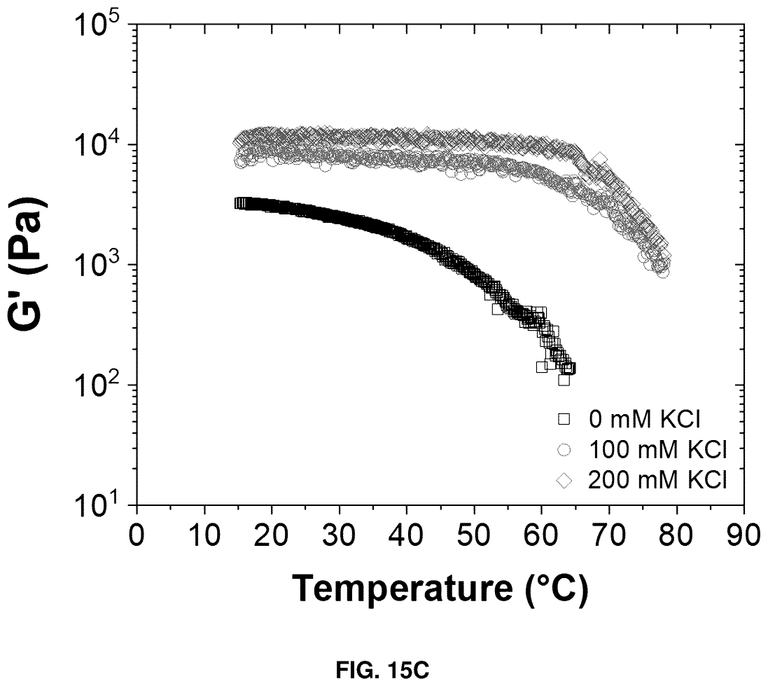

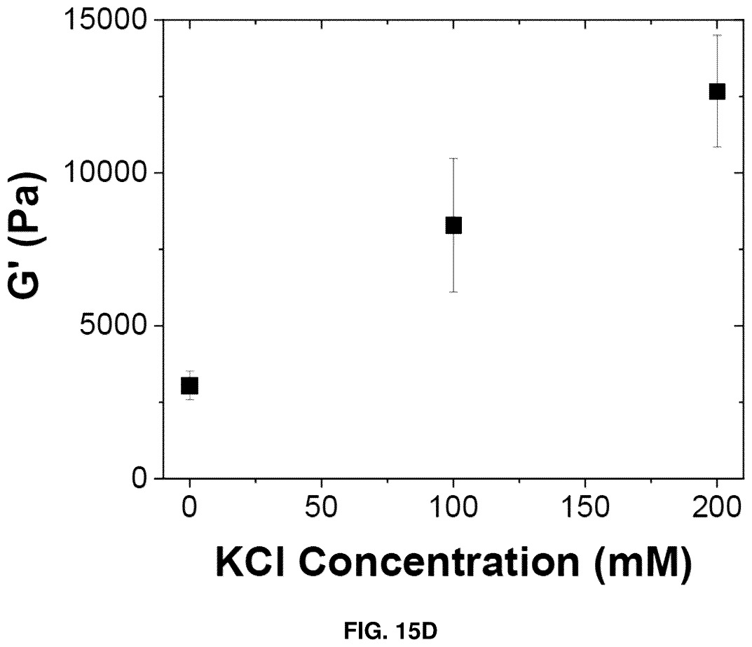

Similarly, as the kappa-carrageenan concentration used to form the hydrogels was raised from 2 to 4 wt % (at a fixed 100 mM KCl concentration), the G′ values increased correspondingly from roughly 5×10³ to 2×10⁴ Pa (FIGS. 15A-15B). These values were comparable to those reported in the literature. Also evident was the effect of KCl on the gel strength. As the KCl concentration was varied from 0 to 200 mM (at a constant 3 wt % kappa-carrageenan concentration), the G′ values again increased (FIGS. 15C-15D).

The compositional effects on the rheology of the thermogelling kappa-carrageenan gels also extended to their heat sensitivity. Namely, the gels formed at higher polymer concentrations began their sharp decline in G′ at slightly higher temperatures (i.e., at ˜70° C. at the 4 wt % concentration rather than at ˜60° C. at lower kappa-carrageenan concentrations; see FIG. 15A). This effect was even more pronounced when the KCl concentration was varied. Gels prepared without KCl exhibited a notable decline in their moduli with temperature even in the 20-30° C. range (with this decrease becoming sharper as the temperature continued to rise). Conversely, gels prepared with 100-200 mM KCl maintained constant G′ values until the temperature reached ˜60-70° C. These trends were qualitatively consistent with prior rheological studies.

### Layer Number Effect on Water/Kappa-Carrageenan-Based Wet Wipe Strength

The tensile strengths of the water-based, kappa-carrageenan wipes increase as the number of the number of base toilet paper layers was raised from two to three (FIG. 16). The average tensile strength of the 2 wt % kappa-carrageenan wipes increased from 40±11 to 78±21 N/m while for wipes prepared from 3 wt % kappa-carrageenan, it increased from 95±17 to 134±18 N/m. Thus, similar to the alcohol wipes prepared using alginate, the kappa-carrageenan-based wet wipe strength increases with the number of base toilet paper layers.

Embossing the Wipes with Patterns

Wipe formation was also achieved using a rolling press, which accelerated and improved the uniform biopolymer solution dispersal throughout the wipe. Moreover, this approach could be used to emboss the wipes with patterns. To this end, K-carrageenan solution was prepared as previously described and applied to the stacked toilet paper layers in a stripe-like pattern (FIG. 18(i)). The K-carrageenan solution-covered toilet paper stacks were then inserted into a 5″×7″ PUPUZAO plastic embossing folder (with a weaving pattern; see FIG. 18 step (ii)), whereupon the embossing folder was pressed between two rollers (spaced roughly 4 mm apart), as shown in FIG. 18 step (iii). This pressing procedure evenly spread the K-carrageenan solution throughout the compressed and embossed toilet paper layers (FIG. 18 step (iv)). To gel the K-carrageenan solution, the resulting wipes were then immersed in the KCl solution as described previously.

### Impact on Biological Wastewater Treatment

In addition to confirming the degradation in their mechanical properties when flushed, the potential impact of both the wet wipes and the eluted biopolymer molecules on the biological wastewater treatment was also examined. One of the most common problems encountered during the biological wastewater treatment is the disturbance of wastewater biomass in response to toxic chemicals, which results in low quality of treated water. Nine batch bioreactors were constructed using 1-L glass flasks with aeration systems and operated with biomass collected from a local wastewater treatment plant. All reactors were fed with raw wastewater collected from a primary clarifier of the same wastewater treatment plant. Using the bioreactors, the changes in the performances of the wastewater treatment process, as well as the quantity and activity of biomass (a.k.a. activated sludge) in the presence of wet-wipes and eluted chemicals (alginate, ethanol, kappa-carrageenan, and KCl), were monitored. Given the low anticipated biopolymer concentrations in the water entering wastewater treatment plants (due to the dilution of the wet wipe degradation products in the toilet, sewer pipes, and wastewater collection systems), two different chemical concentrations (5% and 50% of those produced upon dispersing the wipes in 1 gal of water) were tested by diluting the dispersed degradation products in raw wastewater and biomass. In the summary of these experiments below, these test conditions will be referred to as the 5% and 50% conditions.

The performance of the bioreactors was determined by sludge volume indexes (SVIs) and removal of chemical oxygen demand (COD). The SVI indicates the settleability of biomass and is a commonly monitored parameter in determining the bioreactor performance of the activated sludge process, while the COD is used to estimate the amount of pollutants in wastewater. Further, the mixed liquor suspended solids (MLSS) and mixed liquor volatile suspended solids (MLVSS) were determined for total biomass quantification, and the active biomass quantities were ascertained using adenosine triphosphate (ATP) kits for wastewater biomass (Quench-Gone, Aqueous, LuminaUltra, Canada). Biomass stress index (BSI, a measure of the stress level of microbial community) in the presence dissolved/dispersed wet wipes was calculated from the obtained ATP results as:

$$BSI ( \% ) = \frac{dATP ( ng ATP / mL )}{tATP ( ng ATP / mL )} \times 100 \%$$

where tATP is the total ATP (both its intracellular and extracellular content), while dATP is the dissolved, extracellular ATP.

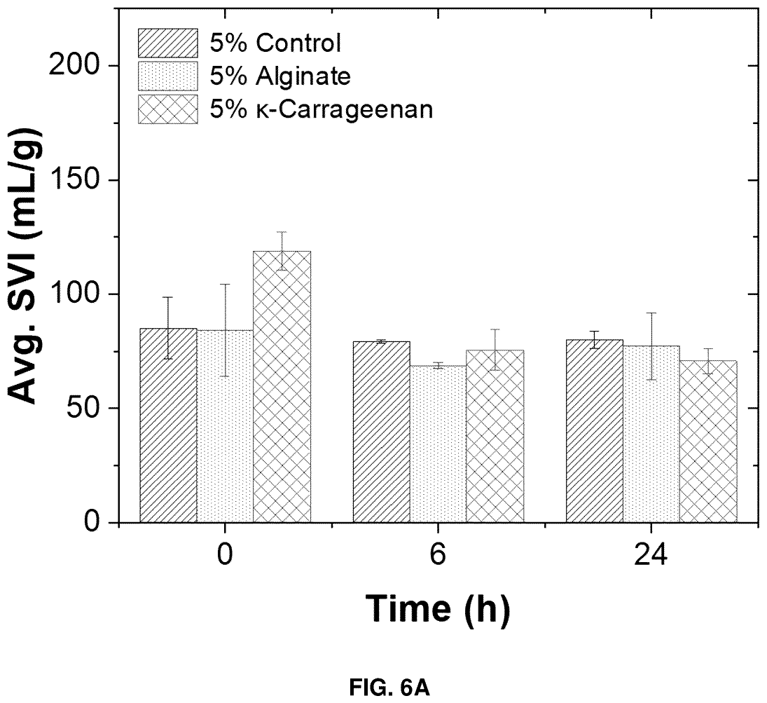

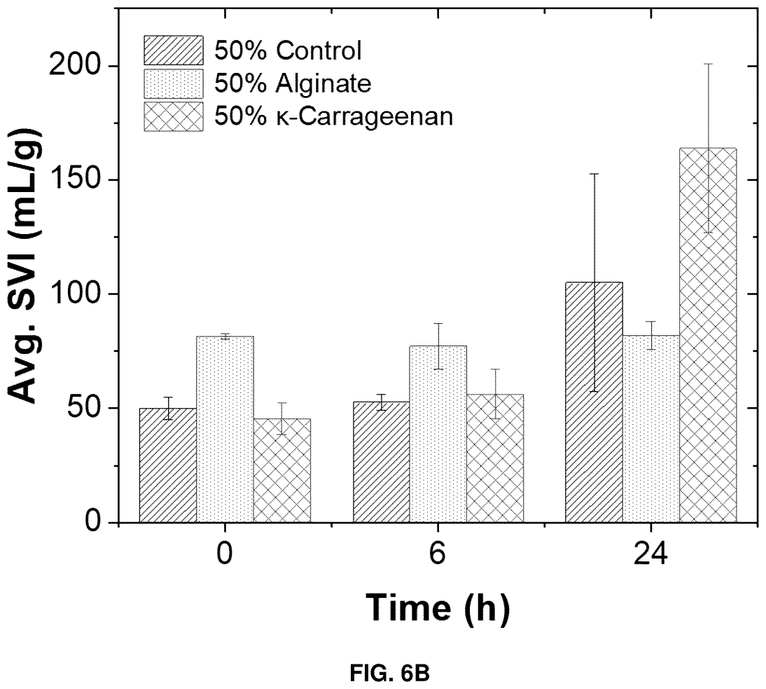

FIGS. 6A-6B display the changes of SVI at different sampling points. There are many different SVI ranges for good biomass settleability, but commonly, an SVI below 100 mL/g is considered good settleability, and a value below 150 mL/g is considered acceptable. Most degradation product samples had average SVI values near or below 100 mL/g, and all conditions tested at the 5% degradation product concentration (which was closer to the highly diluted concentration expected in wastewater treatment bioreactors) produced a SVIs below 150 mL/g. For biomass samples with very high degradation product concentrations (50% of those produced upon dispersing the wipes in 1 gal of water)—which were tested to ascertain whether the wet wipe degradation products can harm when present in extreme concentrations—the average SVI exceeded 150 mL/g when exposed to the kappa-carrageenan-based wipe degradation products for 24 h. Although SVI values increased at this high wipe degradation product concentration, overall, biomass settled quickly (within 30 min), and (since both tested degradation product concentrations likely significantly exceed those that could be present in wastewater treatment plant) the overall SVI values were deemed satisfactory.

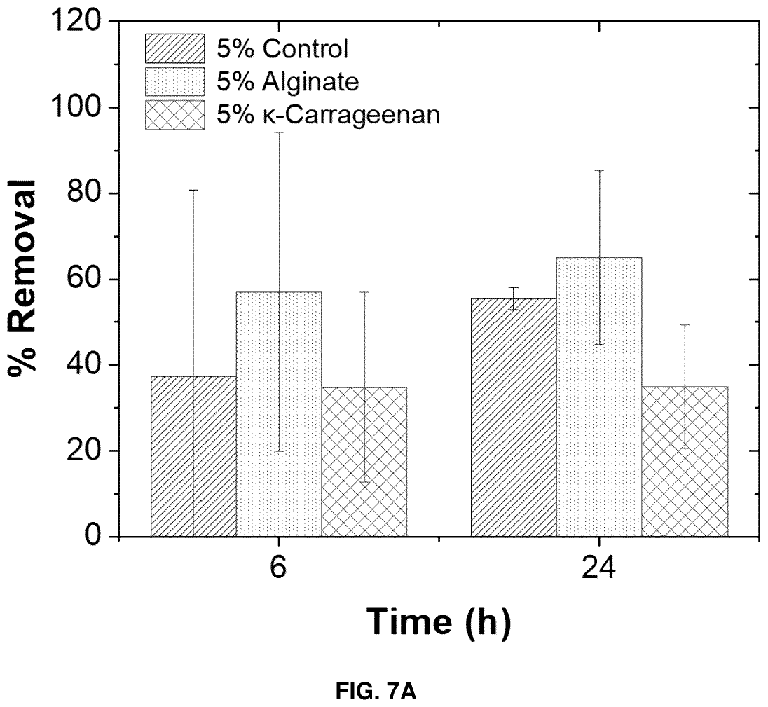

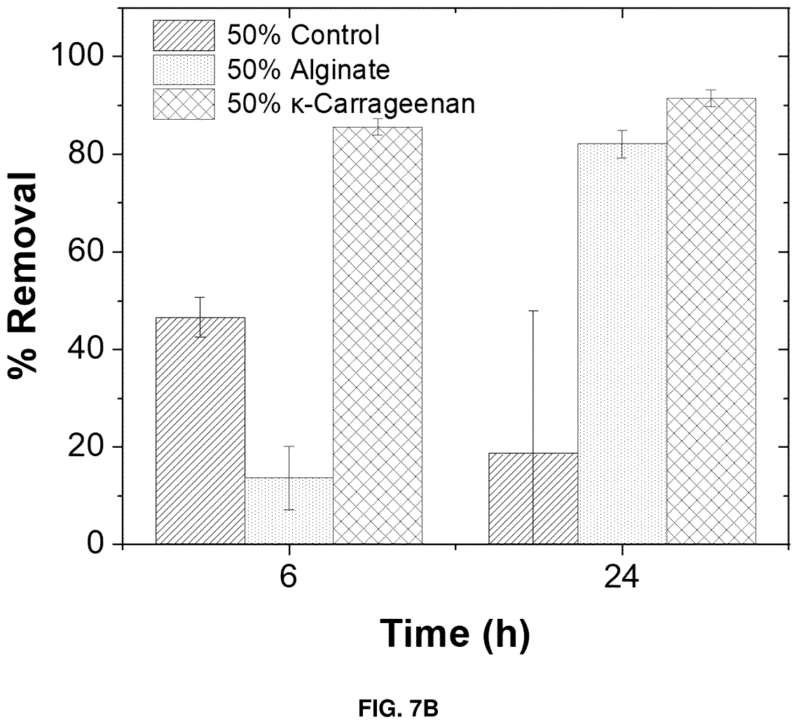

To assess biomass activity and performance of bioreactors for contaminant treatment, COD removal after 6 and 24 hours of contact time was determined (FIGS. 7A-7B). The average COD removal ranged from 14% to 92% and COD levels of bioreactor effluents were in a range of 10-40 mg/L except for the 50% alginate condition. For regulatory compliance, only biochemical oxygen demand (BOD) is currently used, where below 30 mg/L BOD levels (equivalent to 300 mg/L of COD at the 0.1 BOD/COD ratio) are required for the effluents of wastewater bioreactors. All tested samples met the regulatory requirement after 24 h except for samples with high (50% of the initial 1-gal mixture) concentrations of the alginate-based wipes. When concentrated alginate wipe degradation products were tested, initial COD levels were much higher (5,580±210 mg/L of average COD). Despite this result, the bioreactors could rapidly remove most of the COD and achieved 82.1±2.9% removal after 24 h. Moreover, the 50% alginate-based wipe degradation product condition corresponded to a very high degradation product concentration, with a much lower dilution factor than that expected in a wastewater collection and treatment system. In a real wastewater treatment plant, this high level of wet wipe product would not be seen.

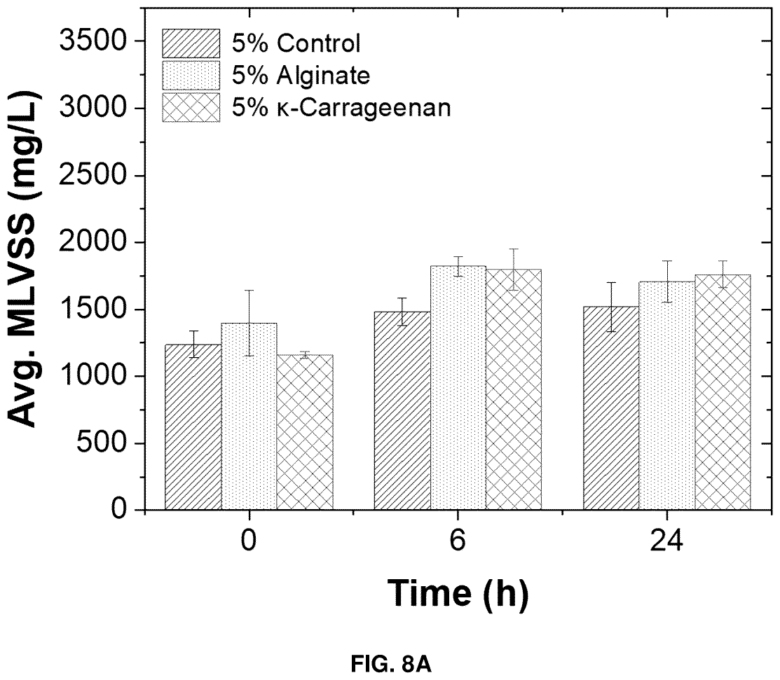

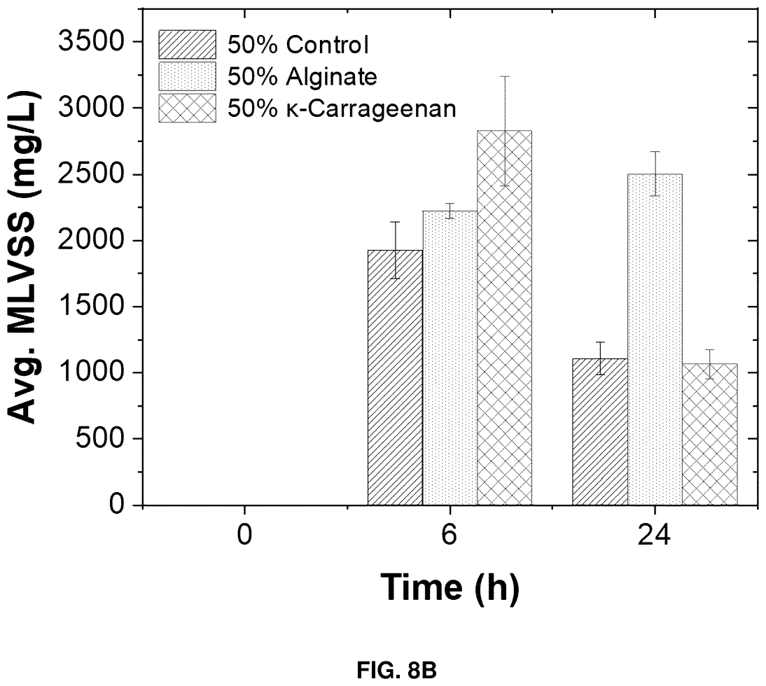

FIGS. 8A-8B show the changes in MLVSS in the bioreactors. MLVSS is the amount of organic and volatile suspended solids and is used as a measure for total biomass in bioreactors for wastewater treatment. During the testing period, there were overall increases of total biomass quantity in the bioreactors with no statistically significant difference between the degradation product and control groups after 24 h. Thus, the chemicals eluted from wet wipes diluted to 5% of their initial dispersion concentrations did not appear to substantially affect the total biomass growth. For the highly concentrated bioreactors with wipe degradation products diluted to 50% of the initial dispersion concentration, however (where there were only two time points), MLVSS trends were inconclusive.

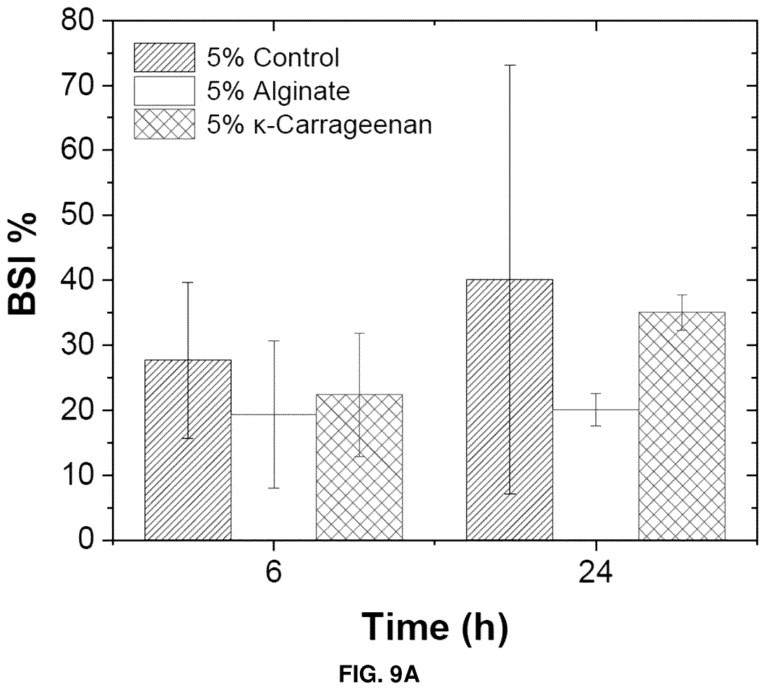

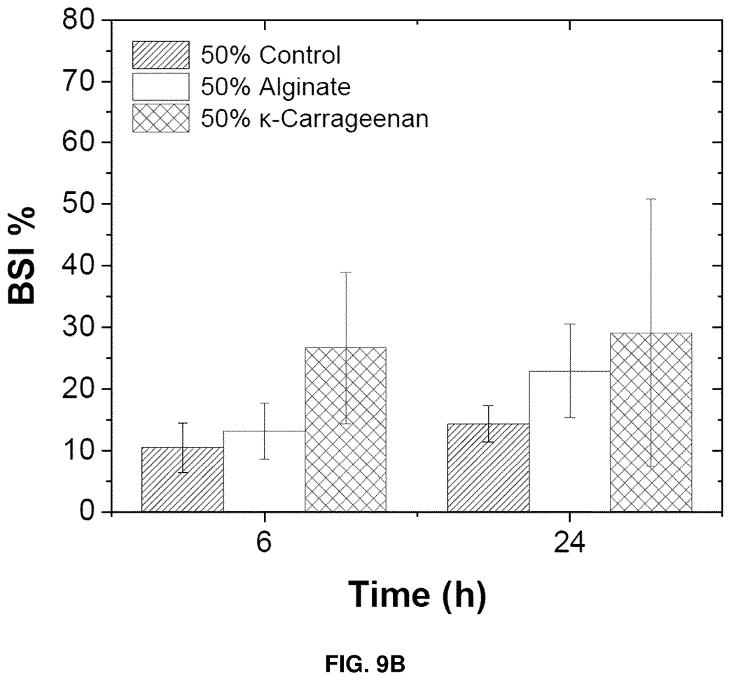

BSIs were also calculated (from the ATP test measurements; see FIG. 9). These values represent the stress level of microbial community in response to physical and chemical stressors. According to the testing kit manufacturer's guidelines (for the LuminUltra QuenchGone21™ Wastewater Test Kit, Product #QG21W-50/QG21Wa-25), BSI values below 30 indicate healthy biomass conditions while values larger than 50 indicate a need for corrective action. The BSI values for all reactors were below 50, except for one 5% control sample at 24 h (where there was one outlier sample). Nonetheless, at the lower concentration, the presence of the wipe degradation products had no adverse effect on the BSI and—although some adverse effect occurs at the very high, 50% concentration (which greatly exceeded those expected in a wastewater treatment plant)—the higher degradation product concentration still maintained the average BSI values below 50.

Overall, the results obtained for the bioreactor performance (i.e., COD and SVI) and biomass quantity/activity (MLVSS and BSI) indicate that the chemicals eluted from wet wipes had virtually no negative impact on biomass and operation of bioreactors at (<5% of the initial 1-gal mixture) degradation product concentrations expected at the wastewater plants.

The wet wipe formulations described in these examples met the success metrics of (1) tensile strength on the order of 100 N/m; (2) transformation back into dispersible toilet paper within 10 min of simulated flushing; and (3) being nondisruptive to wastewater treatment systems.

It has been demonstrated that stimulus-responsive gelation of naturally occurring biopolymers (polysaccharides) enables the generation of wet wipes (both alcoholic and water-based), whose mechanical strength can be rapidly reduced to that of wet toilet paper when they are they are placed in an excess of water. Thus, like toilet paper, these wipes disperse upon being flushed. Moreover, analyses assessing the potential impact of their degradation products on the wastewater treatment process indicate that, at concentrations likely to be encountered at wastewater treatment sites, these biopolymers would not interfere with municipal wastewater treatment. Therefore, by infusing commercial toilet paper with stimulus-responsive biopolymer gels, it is possible to construct wet wipes (both aqueous and alcohol-based) with strengths near or above 100 N/m that when placed in excess water (such as would occur upon flushing) see the gels rapidly dissolve, causing the strength of the wipes to (within 2-3 minutes) drop to levels similar to that of wet toilet paper, thereby allowing them to disperse. Besides their physicochemical properties and compatibility with wastewater treatment bioreactors, the alcohol and water-based wet wipes can be prepared from inexpensive materials. The wet wipes may improve human health/sanitation, reduce costs of private property and municipal infrastructure by reducing damage stemming from flushed wipe-caused clogging, and decrease pollution by protecting and/or rehabilitating wastewater collection/treatment systems and reducing microplastics release.

### CONCLUSIONS

These examples show that interspersing toilet paper with stimulus-responsive biopolymeric gels can generate wet wipes that turn back into (readily dispersible) toilet paper upon being flushed. To this end, two biopolymer-based approaches have been evaluated: one that involved the use of ethanol as a solvent (which generated alcohol wipes that turn into wet toilet paper when flushed), and one that worked with water-based (i.e., aqueous) wet wipes. The ethanol-based approach uses the non-toxic and widely available polysaccharide, alginate, which is water-soluble but forms strong gels when the water is replaced with alcohols. Thus, infusion of toilet paper with aqueous alginate solution followed by its immersion in ethanol forms ethanol-based wet wipes, whose mechanical strength can be tailored (such that their tensile strengths significantly exceed 100 N/m) by varying the alginate concentrations in the aqueous precursor solutions and the number of base toilet paper layers used to form the wipes. The water-based wipes, on the other hand, were formed by interspersing the toilet paper with hot kappa-carrageenan solution, which (especially in the presence of KCl) formed strong gels on cooling.

By tailoring the parent biopolymer solution concentrations, numbers of toilet paper layers, and (in the case of the water/kappa-carrageenan wipes) the KCl concentrations, gel-infused wipes with tensile strengths either near or above the target ˜100 N/m strengths could be obtained with both aqueous and ethanolic solvents. Upon simulated flushing, however (i.e., when the wipes were placed in an excess of water) the gel networks disintegrated and, within minutes, the tensile wipe strength (even when the biopolymers persisted in the gel matrices) became similar to that of wet toilet paper. Since toilet paper is regarded as being safe to flush, this similarity in mechanical strength strongly indicates these toilet paper and biopolymer-based wipes to also have similar flushability. Indeed, this inference was confirmed through flushability tests, where both alcohol/alginate and water/kappa-carrageenan-based wipes indeed cleared the toilet and drain line and then dispersed.

While the wipes tended to be strongest at the highest biopolymer concentrations and numbers of base toilet paper layers, this enhanced mechanical strength came at the expense of other key properties—namely, in diminished flexibility/tactile properties and elevated raw material cost. Thus, there is an optimal biopolymer concentration (3 wt % in these examples) and number of base toilet paper layers (three for the ethanol/alginate-based system and two for the water/kappa-carrageenan-based one). Moreover, analyses assessing the potential impact of their degradation products on the wastewater treatment process indicate that, at concentrations likely to be encountered at wastewater treatment sites, these biopolymers should not interfere with municipal wastewater treatment.

These examples examined flushable wet wipes based on the integration of regular (i.e., flushable) toilet paper with biobased polyelectrolyte gels. The gel matrix reinforced the toilet paper, thus providing the necessary mechanical strength typical of a wet wipe. Two different formulation types were developed to achieve this: one based on ethanol (that generates alcohol wipes) and another water-based (that produced aqueous wet wipes). The ethanolic wipes were prepared by interspersing the toilet paper with alginate gels that were formed by precipitating aqueous alginate in ethanol, while the aqueous wipes were prepared through the KCl-enhanced thermal gelation of kappa-carrageenan. Both wipe types had tensile strengths that increased proportionately with the precursor solution polymer concentration. When exposed to an excess of water, however (i.e., upon simulated flushing), these wipes lost their tensile strengths almost immediately, with the tensile strengths of both wipe types becoming comparable to wet toilet paper within 2-3 min. This rapid transformation in the wet wipe strength reflected the degradation of the gel network and (since toilet paper disperses when flushed) indicates these wipes are—like toilet paper—truly dispersible/flushable. Moreover, this effect was achieved at varying water temperatures (over the entire tested 7-40° C. range), indicating that this technology is insensitive to the toilet/plumbing temperature and, thus, will work in both cold and hot climates. Besides the wipe flushability, the use of biobased polyelectrolytes in their design can reduce the accumulation of harmful microplastics (which are often incorporated into commercial wet wipes) in the environment. Thus, the wet wipes described herein can reduce the clogging in wastewater collection and treatment systems while simultaneously reducing environmental pollution.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A wet wipe comprising:
a water-soluble or dispersible substrate; and
a biopolymer hydrogel composition interspersed on, infused into, or incorporated into, the water-soluble or dispersible substrate, wherein the biopolymer hydrogel composition comprises kappa-carrageenan;
wherein the wet wipe is configured to disperse upon immersion in water with agitation.

2. The wet wipe of claim 1, wherein the biopolymer hydrogel composition is interspersed with, infused into, or incorporated into, a plurality of layers of the water-soluble or dispersible substrate.

3. The wet wipe of claim 2, comprising two layers of the water-soluble or dispersible substrate, wherein the biopolymer hydrogel composition is interspersed between the two layers.

4. The wet wipe of claim 1, wherein the biopolymer hydrogel composition further comprises water, ethanol, acetic acid, dimethyl sulfoxide (DMSO), or polyethylene glycol (PEG).

5. The wet wipe of claim 1, wherein the biopolymer composition further comprises one or more of iota-carrageenan, alginate, chitosan, cellulose, starch, gelatin, xanthan gum, hyaluronic acid, pectin, polylactic acid (PLA), chondroitin sulfate, collagen, elastin, peptidoglycan, or derivatives thereof.

6. The wet wipe of claim 1, wherein the kappa-carrageenan is present in the biopolymer hydrogel composition in an amount of less than 50% by weight.

7. The wet wipe of claim 1, wherein the biopolymer hydrogel composition comprises one or more crosslinkers.

8. The wet wipe of claim 1, wherein the water soluble or dispersible substrate is a paper substrate or a non-woven fabric comprising multi-ply toilet paper, tissue paper, cotton, or rayon.

9. The wet wipe of claim 1, further comprising a monovalent or multivalent salt in the biopolymer hydrogel composition.

10. The wet wipe of claim 9, wherein the monovalent or multivalent salt comprises KCl, NaCl, LiCl, NH₄Cl, NaBr, KBr, NaI, CaCl₂), ZnCl₂, CaCO₃, MgSO₄, sodium acetate, potassium acetate, ammonium acetate, sodium citrate, potassium citrate, ammonium citrate, calcium acetate, or calcium lactate.

11. The wet wipe of claim 1, further comprising a surfactant, a pH adjustment agent, a preservative, or a fragrance.

12. The wet wipe of claim 1, wherein the wet wipe has a tensile strength of about 10² N/m or more, or wherein the wet wipe has a tensile strength of at least about 50 N/m.

13. The wet wipe of claim 1, wherein the wet wipe is free of plastic.

14. The wet wipe of claim 1, wherein the wet wipe is free of alcohol and free of plastic, and the water-soluble or dispersible substrate comprises toilet paper.

15. The wet wipe of claim 1, wherein the biopolymer hydrogel composition comprises less than 50% by weight kappa-carrageenan, about 1-1000 mM of a monovalent salt, and water.

16. A method of making a flushable wet wipe, the method comprising:
adding a warm solution of a carrageenan to a plurality of sheets of toilet paper;
allowing the carrageenan to infuse into the sheets of toilet paper for a period of time, or pressing the solution into the toilet paper, to create infused toilet paper;
allowing the infused toilet paper to cool; and
immersing the infused toilet paper in a solution of a monovalent or multivalent salt to make a flushable wet wipe.

17. The method of claim 16, wherein the carrageenan is kappa-carrageenan.

18. The method of claim 16, wherein the monovalent or multivalent salt comprises KCl, NaCl, LiCl, NH₄Cl, NaBr, KBr, NaI, CaCl₂), ZnCl₂, CaCO₃, MgSO₄, sodium acetate, potassium acetate, ammonium acetate, sodium citrate, potassium citrate, ammonium citrate, calcium acetate, or calcium lactate.

19. The method of claim 16, wherein the solution is pressed into the toilet paper until the solution is uniformly distributed and the toilet paper is flat and smooth.

20. A method of making a flushable wet wipe, the method comprising:
overlaying a first layer of a water dispersible substrate crosswise on a second layer of the water dispersible substrate to form a layer stack;
infusing the layer stack with a solution comprising kappa-carrageenan;
spreading the solution on the layer stack by pressing the layer stack together; and
immersing the layer stack in a salt solution to create a biopolymer hydrogel composition interspersed on, infused into, or incorporated into the layer stack and thereby make a flushable wet wipe.

21. The method of claim 20, wherein a rolling press is used for uniform biopolymer solution dispersal throughout the wipe and to emboss the wipes with patterns.

22. The method of claim 20, wherein the water dispersible substrate is toilet paper.

23. The method of claim 20, wherein the layer stack is pressed together until the solution is uniformly distributed and the layer stack is flat and smooth.
